SEQUENCE LISTING (16 sequences)

SEQ ID NO: 1 - DNA, 767 bp, anthozoa
acttggttttggcttcccggcaaaagaacaacgttaagacgacaactaacaccaaataaatcctgacaatggcttcctttttaaagaagactatgccctttaagacgaccattgaagggacggttaatggccactacttcaagtgtacaggaaaaggagagggcaacccatttgagggtacgcaggaaatgaagatagaggtcatcgaaggaggtccattgccatttgccttccacattttgtcaacgagttgtatgtacggtagtaagaccttcatcaagtatgtgtcaggaattcctgactacttcaagcagtctttccctgaaggttttacttgggaaagaaccacaacctacgaggatggaggctttcttacagctcatcaggacacaagcctagatggagattgcctcgtttacaaggtcaagattcttggtaataattttcctgctgatggccccgtgatgcagaacaaagcaggaagatgggagccagccaccgagatagtttatgaagttgacggtgtcctgcgtggacagtctttgatggcccttaagtgccctggtggtcgtcatctgacttgccatctccatactacttacaggtccaaaaaaccagctagtgccttgaagatgccaggatttcattttgaagatcatcgcatcgagataatggaggaagttgagaaaggcaagtgctataaacagtacgaagcagcagtgggcaggtactgtgatgctgctccatccaagcttggacataactaa

SEQ ID NO: 2 - protein, 232 aa, anthozoa
MASFLKKTMPFKTTIEGTVNGHYFKCTGKGEGNPFEGTQEMKIEVIEGGPLPFAFHILSTSCMYGSKTFIKYVSGIPDYFKQSFPEGFTWERTTTYEDGGFLTAHQDTSLDGDCLVYKVKILGNNFPADGPVMQNKAGRWEPATEIVYEVDGVLRGQSLMALKCPGGRHLTCHLHTTYRSKKPASALKMPGFHFEDHRIEIMEEVEKGKCYKQYEAAVGRYCDAAPSKLGHN

SEQ ID NO: 3 - DNA, 767 bp, anthozoa
acttggttttggcttcccggcaaaagaacaacgttaagacgacaactaacaccaaataaatcctgacaatggcttcctttttaaagaagactatgccctttaagacgaccattgaagggacggttaatggccactacttcaagtgtacaggaaaaggagagggcaacccatttgagggtacgcaggaaatgaagatagaggtcatcgaaggaggtccattgccatttgccttccacattttgtcaacgagttgtatgtacggtagtaagaccttcatcaagtatgtgtcaggaattcctgactacttcaagcagtctttccctgaaggttttacttgggaaagaaccacaacctacgaggatggaggctttcttacagctcatcaggacacaagcctagatggagattgcctcgtttacaaggtcaagattcttggtaataattttcctgctgatggccccgtgatgcagaacaaagcaggaagatgggagccaggcaccgagatagtttatgaagttgacggtgtcctgcgtggacagtctttgatggcccttaagtgccctggtggtcgtcatctgacttgccatctccatactacttacaggtccaaaaaaccagctagtgccttgaagatgccaggatttcattttgaagatcatcgcatcgagataatggaggaagttgagaaaggcaagtgctataaacagtacgaagcagcagtgggcaggtactgtgatgctgctccatccaagcttggacataactaa

SEQ ID NO: 4 - protein, 232 aa, anthozoa
MASFLKKTMPFKTTIEGTVNGHYFKCTGKGEGNPFEGTQEMKIEVIEGGPLPFAFHILSTSCMYGSKTFIKYVSGIPDYFKQSFPEGFTWERTTTYEDGGFLTAHQDTSLDGDCLVYKVKILGNNFPADGPVMQNKAGRWEPGTEIVYEVDGVLRGQSLMALKCPGGRHLTCHLHTTYRSKKPASALKMPGFHFEDHRIEIMEEVEKGKCYKQYEAAVGRYCDAAPSKLGHN

SEQ ID NO: 5 - DNA, 767 bp, anthozoa
acttggttttggcttcccggcaaaagaacaacgttaagacgacaactaacaccaaataaatcctgacaatggcttcctttttaaagaagactatgccctttaagacgaccattgaagggacggttaatggccactacttcaagtgtacaggaaaaggagagggcaacccatttgagggtacgcaggaaatgaagatagaggtcatcgaaggaggtccattgccatttgccttccacattttgtcaacgagttgtatgtacggtagtaagaccttcatcaagtatgtgtcaggaattcctgactacttcaagcagtctttccctgaaggtcttacttgggaaagaaccacaacctacgaggatggaggctttcttacagctcatcaggacacaagcctagatggagattgcctcgtttacaaggtcaagattcttggtaataattttcctgctgatggccccgtgatgcagaacaaagcaggaagatgggagccaggcaccgagatagtttatgaagttgacggtgtcctgcgtggacagtctttgatggcccttaagtgccctggtggtcgtcatctgacttgccatctccatactacttacaggtccaaaaaaccagctagtgccttgaagatgccaggatttcattttgaagattaccgcatcgagataatggaggaagttgagaaaggcaagtgctataaacagtacgaagcagcagtgggcaggtactgtgatgctgctccatccaagcttggacataactaa

SEQ ID NO: 6 - protein, 232 aa, anthozoa
MASFLKKTMPFKTTIEGTVNGHYFKCTGKGEGNPFEGTQEMKIEVIEGGPLPFAFHILSTSCMYGSKTFIKYVSGIPDYFKQSFPEGLTWERTTTYEDGGFLTAHQDTSLDGDCLVYKVKILGNNFPADGPVMQNKAGRWEPGTEIVYEVDGVLRGQSLMALKCPGGRHLTCHLHTTYRSKKPASALKMPGFHFEDYRIEIMEEVEKGKCYKQYEAAVGRYCDAAPSKLGHN

SEQ ID NO: 7 - DNA, 760 bp, Anthozoa
accatttgctttggttccttggcaaacgaaagtttagacgaaaactgacccaaattacatcctcctgatccttaccatggctggtttgttgaaagaaagtatgcgcatcaagatgtacatggaaggcacggttaatggccattatttcaagtgtgaaggagagggagacggcaacccatttacaggtacgcagagcatgaggattcatgtcaccgaaggggctccattaccatttgccttcgacattttggcaccgtgttgtgagtacggcagcaggacctttgtccaccatacggcagagattcccgatttcttcaagcagtctttccctgaaggctttacttgggaaagaaccacaacctatgaagatggaggcattcttactgctcatcaggacacaagcctggaggggaactgccttatatacaaggtgaaagtccttggtaccaattttcctgctgatggccccgtgatgaagaacaaatcaggaggatgggagccatgcactgaggtggtttatccagagaatggtgtcctgtgtggacgtaatgtgatggcccttaaagtcggtgatcgtcgtttgatctgccatctctatacttcttacaggtccaagaaagcagtccgtgccttgacaatgccaggatttcattttacagacatccgccttcagatgccgaggaaaaagaaagacgagtactttgaactgtacgaagcatctgtggctaggtacagtgatcttcctgaaaaagcaaattga

SEQ ID NO: 8 - protein, 215 aa, Anthozoa
MAGLLKESMRIKMYMEGTVNGHYFKCEGEGDGNPFTGTQSMRIHVTEGAPLPFAFDILAPCCEYGSRTFVHHTAEIPDFFKQSFPEGFTWERTTTYEDGGILTAHQDTSLEGNCLIYKVKVLGTNFPADGPVMKNKSGGWEPCTEVVYPENGVLCGRNVMALKVGDRRLICHLYTSYRSKKAVRALTMPGFHFTDIRLQMPRKKKDEYFELYEAS

SEQ ID NO: 9 - DNA, 760 bp, Anthozoa
accatttgctttggttccttggcaaacgaaagtttagacgaaaactgacccaaattacatcctcctgatccttaccatggctggtttgttgaaagaaagtatgcgcatcaagatgtacatggaaggcacggttaatggccattatttcatgtgtgaaggagagggagacggcaacccatttacaggtacgcagagcatgaggattcatgtcaccgaaggggctccattaccatttgccttcgacattttggcaccgtgttgtgagtacggcagcaggacctttgtccaccatacggcagagattcccgatttcttcaagcagtctttccctgaaggctttacttgggaaagaaccacaacctatgaagatggaggcattcttactgctcatcaggacacaagcctggaggggaactgccttatatacaaggtgaaagtccttggtaccaattttcctgctgatggccccgtgatgaagaacaaatcaggaggatgggagccatgcactgaggtggtttatccagagaatggtgtcctgtgtggacgtgcagtgatggcccttaaagtcggtgatcgtcgtttgatctgccatctctatacttcttacaggtccaagaaagcagtccgtgccttgacaatgccaggatttcattttacagacatccgccttcagatgccgaggaaaaagaaagacgagtactttgaactgtacgaagcatctgtggctaggtacagtgatcttcctgaaaaagcaaattga

SEQ ID NO: 10 - protein, 227 aa, Anthozoa
MAGLLKESMRIKMYMEGTVNGHYFMCEGEGDGNPFTGTQSMRIHVTEGAPLPFAFDILAPCCEYGSRTFVHHTAEIPDFFKQSFPEGFTWERTTTYEDGGILTAHQDTSLEGNCLIYKVKVLGTNFPADGPVMKNKSGGWEPCTEVVYPENGVLCGRAVMALKVGDRRLICHLYTSYRSKKAVRALTMPGFHFTDIRLQMPRKKKDEYFELYEASVARYSDLPEKAN

SEQ ID NO: 11 - DNA, 760 bp, Anthozoa
accatttgctttggttccttggcaaacgaaagtttagacgaaaactgacccaaattacatcctcctgatccttaccatggctggtttgttgaaagaaagtatgcgcatcaagatgtacatggaaggcacggttaatggccattatttcatgtgtgaaggagagggagacggcaacccatttacaggtacgcagagcatgaggattcatgtcaccgaaggggctccattaccatttgccttcgacattttggcaccgtgttgtgagtacggcagcaggacctttgtccaccatacggcagagattcccgatttcttcaagcagtctttccctgaaggctttacttgggaaagaaccacaacctatgaagatggaggcattcttactgctcatcaggacacaagcctggaggggaactgccttatatacaaggtgaaagtccttggtaccaattttcctgctgatggccccgtgatgaagaacaaatcaggaggatgggagccatgcactgaggtggtttatccagagaatggtgtcctgtgtggacgtggtgtgatggcccttaaagtcggtgatcgtcgtttgatctgccatctctatacttcttacaggtccaagaaagcagtccgtgccttgacaatgccaggatttcattttacagacatccgccttcagatgccgaggaaaaagaaagacgagtactttgaactgtacgaagcatctgtggctaggtacagtgatcttcctgaaaaagcaaattga

SEQ ID NO: 12 - protein, 227 aa, Anthozoa
MAGLLKESMRIKMYMEGTVNGHYFMCEGEGDGNPFTGTQSMRIHVTEGAPLPFAFDILAPCCEYGSRTFVHHTAEIPDFFKQSFPEGFTWERTTTYEDGGILTAHQDTSLEGNCLIYKVKVLGTNFPADGPVMKNKSGGWEPCTEVVYPENGVLCGRGVMALKVGDRRLICHLYTSYRSKKAVRALTMPGFHFTDIRLQMPRKKKDEYFELYEASVARYSDLPEKAN

SEQ ID NO: 13 - DNA, 760 bp, Anthozoa
accatttgctttggttccttggcaaacgaaagtttagacgaaaactgacccaaattacatcctcctgatccttaccatggctggtttgttgaaagaaagtatgcgcatcaagatgtacatggaatgcacggttaatggccattatttcaagtgtgaaggagagggagacggcaacccatttgcaggtacgcagagcatgaggattcatgtcaccgaaggggctccattaccatttgccttcgacattttggcaccgtgttgtgagtacggcagcaggacctttgtccaccatacggcagagattcccgatttcttcaagcagtctttccctgaaggctttacttgggaaagaaccacaacctatgaagatggaggcattcttactgctcatcaggacacaagcctggaggggaactgccttatatacaaggtgaaagtccatggtaccaattttcctgctgatggccccgtgatgaagaacaaatcaggaggatgggagccagcaactgaggtggtttatccagagaatggtgtcctgtgtggacgtggcgtgatggcccttaaagtcggtgatcgtcatttgatctgccatcactatacttcttacaggtccaagaaagtagtccgtgccttgacaatgccaggatttcattttacagaccaccgccttcagatgctgaggaaagagaaagacgagtactttgaactgtacgaagcatctgtggctaggtacagtgatcttcctgaaaaagcaaattga

SEQ ID NO: 14 - protein, 227 aa, Anthozoa
MAGLLKESMRIKMYMECTVNGHYFKCEGEGDGNPFAGTQSMRIHVTEGAPLPFAFDILAPCCEYGSRTFVHHTAEIPDFFKQSFPEGFTWERTTTYEDGGILTAHQDTSLEGNCLIYKVKVHGTNFPADGPVMKNKSGGWEPATEVVYPENGVLCGRGVMALKVGDRHLICHHYTSYRSKKVVRALTMPGFHFTDHRLQMLRKEKDEYFELYEASVARYSDLPEKAN

SEQ ID NO: 15 - DNA, 760 bp, Anthozoa
accatttgctttggttccttggcaaacgaaagtttagacgaaaactgacccaaattacatcctcctgatccttaccatggctggtttgttgaaagaaagtatgcgcatcaagatgtacatggaaggcacggttaatggccattatttcaagtgtgaaggagagggagacggcaacccatttgcaggtacgcagagcatgaggattcatgtcaccgaaggggctccattaccatttgccttcgacattttggcaccgtgttgtgagtacggcagcaggacctttgtccaccatacggcagagattcccgatttcttcaagcagtctttccctgaaggctttacttgggaaagaaccacaacctatgaagatggaggcattcttactgctcatcaggacacaagcctggaggggaactgccttatatacaaggtgaaagtccttggtaccaattttcctgctgatggccccgtgatgaagaacaaatcaggaggatgggagccaagcactgaggtggtttatccagagaatggtgtcctgtgtggacgtagcgtgatggcccttaaagtcggtgatcgtcgtttgatctgccatcactatacttcttacaggtccaagaaagcagtccgtgccttgacaatgccaggatttcattttacagaccaccgccttcagatgcggaggaaagagaaagacgagtactttgaactgtacgaagcatctgtggctaggtacagtgatcttcctgaaaaagcaaattga

SEQ ID NO: 16 - protein, 227 aa, Anthozoa
MAGLLKESMRIKMYMEGTVNGHYFKCEGEGDGNPFAGTQSMRIHVTEGAPLPFAFDILAPCCEYGSRTFVHHTAEIPDFFKQSFPEGFTWERTTTYEDGGILTAHQDTSLEGNCLIYKVKVLGTNFPADGPVMKNKSGGWEPSTEVVYPENGVLCGRSVMALKVGDRRLICHHYTSYRSKKAVRALTMPGFHFTDHRLQMRRKEKDEYFELYEASVARYSDLPEKAN

(19) United States
(12) Patent Application Publication
(10) Pub. No.: US 2003/0092884 A1
(43) Pub. Date: May 15, 2003

(54) Kindling fluorescent proteins and methods for their use

(76) Inventors: Sergey A. Lukyanov, Moscow (RU); Konstantin Lukyanov, Moscow (RU); Dmitry Chudakov, Moscow (RU)

Correspondence Address: BOZICEVIC, FIELD & FRANCIS LLP, 200 MIDDLEFIELD RD, SUITE 200, MENLO PARK, CA, 94025, US

(21) Appl. No.: 10/155,809

(22) Filed: May 24, 2002

Related U.S. Application Data
(60) Provisional application No. 60/293,752, filed on May 25, 2001.
(60) Provisional application No. 60/329,176, filed on Oct. 11, 2001.

(51) Int. Cl. C12Q 1/00; C07H 21/04; C07K 14/435; C12P 21/02; C12N 5/06

(52) U.S. Cl. 530/350; 435/69.1; 435/320.1; 435/325; 536/23.5; 435/4

(57) ABSTRACT

Kindling fluorescent protein compositions and nucleic acids encoding the same, as well as methods for using the same, are provided. The kindling fluorescent proteins are characterized in that they become brightly fluorescent proteins, from an initial non-fluorescent or low fluorescent state, upon exposure to a kindling stimulus, which fluorescent state may be reversible or irreversible. The subject protein/nucleic acid compositions find use in labeling protocols, e.g., in labeling proteins, organelles, cells and organisms, etc., in a variety of different types of applications. Also provided are systems and kits for use in practicing such applications.

### CROSS-REFERENCE To RELATED APPLICATIONS

Pursuant to 35 U.S.C. §119(e), this application claims priority to the filing date of U.S. Provisional Patent Application Serial No. 60/293,752 filed May 25, 2001; and to the filing date of U.S. Provisional Patent Application Serial No. 60/329,176 filed Oct. 11, 2001; the disclosures of which applications are herein incorporated by reference.

The present invention also claims priority to U.S. application Ser. No. 10/006,922 filed on Dec. 4, 2001; which application in turn claims priority to U.S. application Ser. No. 09/210,330 filed Dec. 11, 1998; and to U.S. application Ser. No. 09/976,673 filed Oct. 12, 2001; the disclosures of which are herein incorporated by reference.

### INTRODUCTION

1. Field of the Invention

The field of this invention is fluorescent proteins and the use thereof for labeling protocols.

2. Background of the Invention

Labeling protocols play a prominent role in many biochemistry, molecular biology, medical diagnostic and related applications. Labeling protocols depend on labeling or marking an entity of interest, e.g., a protein, organelle, cell, or organism, etc., with a detectable moiety, i.e., marker or label. A variety of different labels have been developed, including radiolabels, chromolabels, fluorescent labels, chemiluminescent labels, etc. Of particular interest in many applications are fluorescent protein labels. Examples of such fluorescent protein labels include the Aequorea victorea green fluorescent protein and mutants thereof, and the Reef Coral Fluorescent Proteins, as described in Matz, M. V., et al. (1999) Nature Biotechnol., 17:969-973.

Tracking intracellular and intercellular protein movement is important in many applications, including research applications, drug development applications, and diagnostic applications, such as imaging. One of the most common techniques currently in use to track intracellular protein movement is photobleaching. In photobleaching, a discrete area of a cell containing fluorescent molecules (e.g., fluorescently tagged proteins) is irradiated to quench the fluorescence in the discrete area. Protein movement can be traced by following influx of fluorescently-labeled proteins from outside of the bleached region into the bleached region. Alternatively, protein movement can be traced by monitoring a reduction in fluorescence in an unbleached area of the cell, as bleached proteins leave the bleached region and dilute out fluorescence in an unbleached region. Two prevalent photobleaching techniques are fluorescence recovery after photobleaching (FRAP) and fluorescence loss in photobleaching (FLIP). FRAP is used to estimate the turnover rates of molecules by the rate of influx of a bleached region by unbleached molecules from areas surrounding the bleached region. FLIP is based on monitoring the loss of fluorescence outside a repeatedly bleached region. FLIP studies show continuity of transport between different populations of fluorophores. A similar technique, fluorescent localization after photobleaching (FLAP), in which the molecule to be located carries two fluorophores: one to be photobleached and the other to act as a reference label, has also been suggested. There are, however, certain disadvantages of using photobleaching. For example, the intense laser light used in the photobleaching step can be very damaging to cells and can alter subcellular structures. Damage to a cell can alter the normal movement of a protein within the cell. Furthermore, photobleaching is only an indirect, rather than a direct, means of monitoring protein movement and thus does not allow one to trace directly the major pathways and velocity of a protein's movement from a given point in a cell. Interpretation of the results of photobleaching experiments is prone to artifact, as a decrease in light may be due, not to protein movement, but to other processes, such as protein degradation.

As such, there is a need in the art for improved methods and reagents for use in labeling in general, and labeling for trafficking applications in particular. Of particular interest would be the development of a photoactivatable fluorescent protein. The present invention satisfies this need.

### Relevant Literature

U.S. patents of interest include: U.S. Pat. Nos. 6,066,476; 6,020,192; 5,985,577; 5,976,796; 5,968,750; 5,968,738; 5,958,713; 5,919,445; 5,874,304; and 5,491,084. International Patent Publications of interest include: WO 00/46233; WO 99/49019; and DE 197 18 640 A. The FRAP technique is described in Misteli et al. (2000) Nature 408:877-881; Snaar et al. (2000) J. Cell. Biol. 151:653-662; Dundr et al. (2000) J. Cell Biol. 150:433-446; Estes et al. (2000) J. Neurogenet. 13 ;233-255; Vos et al. (2000) Curr. Biol. 10:1-7; and Gurskaya et al. (2001) FEBS Lett. (in press). The FLIP technique is described in White and Stelzer (1999) Trends Cell Biol. 9:61-65. The FLAP technique is described in Dunn et al. (2002) J. Microscop. 205:109-112.

### SUMMARY OF THE INVENTION

Kindling fluorescent protein compositions and nucleic acids encoding the same, as well as methods for using the same, are provided. The kindling fluorescent proteins are characterized in that they become brightly fluorescent proteins, from an initial non-fluorescent or low fluorescent state, upon exposure to a kindling stimulus, which fluorescent state may be transient or permanent. The subject protein/nucleic acid compositions find use in labeling protocols where an entity of interest is labeled or marked, e.g., in labeling proteins, organelles, cells and organisms, etc., in a variety of different types of applications. Also provided are systems and kits for use in practicing such applications.

### BRIEF DESCRIPTIONS OF THE FIGURES

FIG. 1 provides the nucleotide and amino acid sequences (SEQ ID NOs:01 and 02, respectively) of asFP595 (asCP).

FIG. 2 provides the nucleotide and amino acid sequences (SEQ ID NOs:03 and 04, respectively) of the A148G mutant (i.e., KFP1) of AsFP595.

FIG. 3 provides the nucleotide and amino acid sequences (SEQ ID NOs:05 and 06, respectively of) of the F90L, A148G, H203Y mutant of AsFP595.

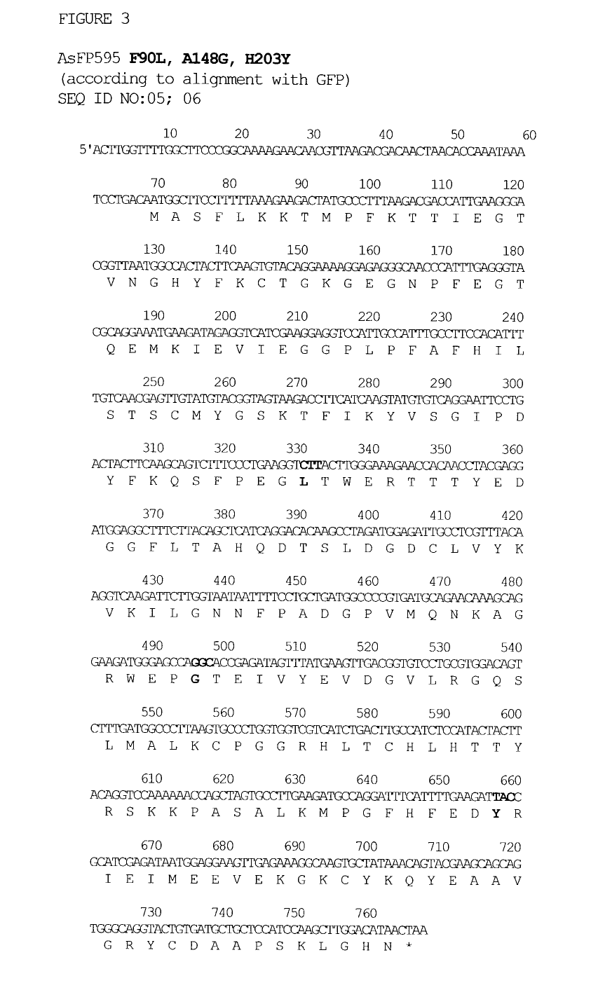

FIG. 4 provides the nucleotide and amino acid sequences (SEQ ID NOs:07 and 08, respectively) of the Heteractis crispa chromoprotein.

FIG. 5 provides the nucleotide and amino acid sequences (SEQ ID NOs:09 and 10, respectively) of the K28M, N165A mutant of Heteractis crispa chromoprotein.

FIG. 6 provides the nucleotide and amino acid sequences (SEQ ID NOs:11 and 12, respectively) of the K28M, N165G mutant of Heteractis crispa chromoprotein.

FIG. 7 provides the nucleotide and amino acid sequences (SEQ ID NOs:13 and 14, respectively) of the G20C, T39A, L126H, C148A, N165G, R176H, L181 H, A190V, I203H, P208L, K211 E mutant of Heteractis crispa chromoprotein.

FIG. 8 provides the nucleotide and amino acid sequences (SEQ ID NOs:15 and 16, respectively) of the T39A, C148S, N165S, L181H, 1203H, P208R, K211 E mutant of Heteractis crispa chromoprotein.

FIGS. 9A-D depict the use of a KFP to study cell migration during embryogenesis.

FIGS. 10A-G depict the use of a KFP to study migration of a mitochondrion.

### DEFINITIONS

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” (B. D. Hames & S. J. Higgins eds. (1985)); “Transcription and Translation” (B. D. Hames & S. J. Higgins eds. (1984)); “Animal Cell Culture” (R. I. Freshney, ed. (1986)); “Immobilized Cells and Enzymes” (IRL Press, (1986)); B. Perbal, “A Practical Guide To Molecular Cloning” (1984).

A “vector” is a replicon, such as plasmid, phage or cosmid, to which another DNA segment may be attached so as to bring about the replication of the attached segment.

The terms “polynucleotide” and “nucleic acid molecule” are used interchangeably herein to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art. The term “polynucleotide” includes double-stranded DNA found, inter alia, in linear DNA molecules (e.g., restriction fragments), viruses, plasmids, and chromosomes.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules, such as methylated nucleic acid molecules and nucleic acid molecule analogs. Analogs of purines and pyrimidines are known in the art. Nucleic acids may be naturally occurring, e.g. DNA or RNA, or may be synthetic analogs, as known in the art. Such analogs may be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O- phosphorothioate, 3′-CH2-5′-O-phosphonate and 3′-NH-5′-O-phosphoroamidate. Peptide nucleic acids replace the entire ribose phosphodiester backbone with a peptide linkage. A DNA “coding sequence” is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence may be located 3′ to the coding sequence.

As used herein, the term “hybridization” refers to the process of association of two nucleic acid strands to form an antiparallel duplex stabilized by means of hydrogen bonding between residues of the opposite nucleic acid strands. A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at http://ww.ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif. USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970). Of interest is the BestFit program using the local homology algorithm of Smith Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis. USA. Another program of interest is the FastDB algorithm. FastDB is described in Current Methods in Sequence Comparison and Analysis, Macromolecule Sequencing and Synthesis, Selected Methods and Applications, pp.127-149, 1988, Alan R. Liss, Inc. Percent sequence identity is calculated by FastDB based upon the following parameters:

Mismatch Penalty: 1.00;

Gap Penalty: 1.00;

Gap Size Penalty: 0.33; and

Joining Penalty: 30.0.

One parameter for determining percent sequence identity is the “percentage of the alignment region length” where the strongest alignment is found. The percentage of the alignment region length is calculated by counting the number of residues of the individual sequence found in the region of strongest alignment. This number is divided by the total residue length of the target or query polynucleotide sequence to find a percentage. An example is shown below:

| Target sequence: | GCGCGAAATACTCACTCGAGG |
|---|---|
| | \| \|\|\| \|\|\|\| \|\|\| |
| Query sequence: | TATAGCCCTAC.CACTAGAGTCC |
| | 1 5 10 15 |

The region of alignment begins at residue 9 and ends at residue 19. The total length of the target sequence is 20 residues. The percent of the alignment region length is 11 divided by 20 or 55%, for example.

Percent sequence identity is calculated by counting the number of residue matches between the target and query polynucleotide sequence and dividing total number of matches by the number of residues of the target or query sequence found in the region of strongest alignment. For the example above, the percent identity would be 10 matches divided by 11 residues, or approximately, 90.9% The percent of the alignment region length is typically at least about 55% of total length of the sequence, more typically at least about 58%, and even more typically at least about 60% of the total residue length of the sequence. Usually, percent length of the alignment region can be as great as about 62%, more usually as great as about 64% and even more usually as great as about 66%.

The term “oligonucleotide” refers to a short (under 100 bases in length) nucleic acid molecule.

The terms “DNA regulatory sequences”, and “regulatory elements”, used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding regions responsible for the binding of RNA polymerase. Eukaryotic promoters will often, but not always, contain “TATA” boxes and “CAT” boxes. Various promoters, including inducible promoters, may be used to drive expression.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

A “clone” is a population of cells derived from a single cell or common ancestor by mitosis. A “cell line” is a clone of a primary cell that is capable of stable growth in vitro for many generations.

A “heterologous” region of the DNA construct is an identifiable segment of DNA within a larger DNA molecule that is not found in association with the larger molecule in nature. Thus, e.g., when the heterologous region encodes a mammalian gene, the gene will usually be flanked by DNA that does not flank the mammalian genomic DNA in the genome of the source organism. In another example, heterologous DNA includes coding sequence in a construct where portions of genes from two different sources have been brought together so as to produce a fusion protein product. Allelic variations or naturally-occurring mutational events do not give rise to a heterologous region of DNA as defined herein.

As used herein, the term “reporter gene” refers to a coding sequence attached to heterologous promoter or enhancer elements and whose product may be assayed easily and quantifiably when the construct is introduced into tissues or cells.

The amino acids described herein are preferred to be in the “L” isomeric form. The amino acid sequences are given in one-letter code (A: alanine; C: cysteine; D: aspartic acid; E: glutamic acid; F: phenylalanine; G: glycine; H: histidine; I: isoleucine; K: lysine; L: leucine; M: methionine; N: asparagine; P: proline; Q: glutamine; R: arginine; S: serine; T: threonine; V: valine; W: tryptophan; Y: tyrosine; X: any residue). NH₂ refers to the free amino group present at the amino terminus of a polypeptide. COOH refers to the free carboxyl group present at the carboxyl terminus of a polypeptide. In keeping with standard polypeptide nomenclature, J. Biol. Chem., 243 (1969), 3552-59 is used.

The term “immunologically active” defines the capability of the natural, recombinant or synthetic chromo/fluorescent protein, or any oligopeptide,thereof, to induce a specific immune response in appropriate animals or cells and to bind with specific antibodies. As used herein, “antigenic amino acid sequence” means an amino acid sequence that, either alone or in association with a carrier molecule, can elicit an antibody response in a mammal. The term “specific binding,” in the context of antibody binding to an antigen, is a term well understood in the art and refers to binding of an antibody to the antigen to which the antibody was raised, but not other, unrelated antigens.

### DETAILED DESCRIPTION OF THE INVENTION

Kindling fluorescent protein compositions and nucleic acids encoding the same, as well as methods for using the same, are provided. The kindling fluorescent proteins (i.e., KFPs) are characterized in that they become brightly fluorescent proteins, from an initial non-fluorescent or low fluorescent state, upon exposure to a kindling stimulus, which fluorescent state may be transient or permanent. The subject protein/nucleic acid compositions find use in labeling protocols where an entity of interest is labeled or marked, e.g., in labeling proteins, organelles, cells and organisms, etc., in a variety of different types of applications. Also provided are systems and kits for use in practicing such applications.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a kindling fluorescent protein” includes a plurality of such proteins and reference to “the method” includes reference to one or more cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, the present invention provides kindling fluorescent protein protein and nucleic acid compositions, as well as methods for using the same and systems and kits for practicing the subject methods. In further describing the subject invention, each of these features is described separately below.

### Kindling Fluorescent Protein Compositions

The kindling fluorescent proteins of the subject invention are proteins that are photoactivatable fluorescent proteins, by which is meant that the kindling fluorescent proteins are proteins that go from a first substantially non-fluorescent or non-fluorescent state to a second fluorescent state in response to exposure to a kindling stimulus, where the kindling stimulus is light of sufficient intensity, wavelength and duration to kindle the protein into a fluorescent protein. “Kindling” as used herein refers to the process whereby a nonfluorescent or weakly fluorescent, i.e., substantially non fluorescent, protein is subjected to a kindling stimulus so that it becomes brightly fluorescent. A given protein is considered to be substantially non-fluorescent or non-fluorescent if any quantum yield of fluorescence detected from the protein upon excitation does not exceed about 0.001-0.005. Brightly fluorescent means that a protein has a minimal quantum yield of fluorescence as determined using the above assay of at least about 0.01, usually at least about 0.04-0.1. Where a given protein is substantially non-fluorescent prior to kindling but does have a minimal amount of detectable fluorescence as determined using the assay referenced above, the magnitude of fluorescence increase following kindling is generally at least several times—e.g., from 1.1-fold to 10,000-fold, from 5-fold to 5000-fold, from 10-fold to 1000-fold, or from 100-fold to 500-fold brighter; in some embodiments from 10-fold to 100-fold brighter.

As indicated above, the kindling fluorescent proteins are photoactivatable such that they go from a first substantially non-fluorescent, if not non-fluorescent, state to a second fluorescent state upon subjection or exposure to a kindling stimulus. The kindling stimulus is light of a sufficient wavelength, intensity and duration to produce change the protein from the first to the second state. In other words, the kindling stimulus is light of kindling wavelength, kindling intensity and kindling duration to change the protein from the first to the second state.

The kindling wavelength of light varies depending on the particular kindling proteins, and includes, but is not limited to, wavelengths in the visible spectrum, e.g., red, orange, yellow, green, blue, indigo, and violet; ultraviolet light; and infrared light. Thus, in many embodiments, a kindling wavelength of light is in the range of from about 190 nm to about 200 nm, from about 200 nm to about 300 nm, from about 300 nm to about 400 nm, from about 380 nm to about 400 nm, from about 400 nm to about 430 nm, from about 430 nm to about 500 nm, from about 500 nm to about 560 nm, from about 560 nm to about 620 nm, from about 620 nm to about 700 nm, from about 700 nm to about 1.5 μm, from about 1.5 μm to about 20 μm, or from about 20 μm to about 1000 μm. As such, the kindling wavelength may range from about 190 nm to about 1000 μm, sometimes about 200 nm to about 1.5 μm and sometimes from about 300 nm to about 700 nm. The kindling wavelength may be a single wavelength or two or more different wavelengths.

The kindling fluorescent protein is exposed to the kindling wavelength for a kindling duration, i.e., discrete period of time, e.g., from about 0.001 second to about 0.005 second, from about 0.005 second to about 0.01 second, from about 0.01 second to about 0.05 second, from about 0.05 second to about 0.1 second, from about 0.1 second to about 0.5 second, from about 0.5 second to about 1 second, from about 1 second to about 5 seconds, from about 5 seconds to about seconds, from about 10 seconds to about 15 seconds, from about 15 seconds to about 30 seconds, or from about 30 seconds to about 60 seconds, from about 1 minute to about 5 minutes, or from about 5 minutes to about 10 minutes, or longer. As such, the length of time may range from about 0.001 second to 10 minutes or longer, sometimes from about 0.1 seconds to about 60 seconds. The length of time to which the protein is exposed to a kindling wavelength of light depends, in part, on the intensity of the kindling light. Thus, in general, the higher the intensity of light, the shorter the time of exposure that is required to kindle the protein (i.e., to induce the conformation change that allows the protein to fluoresce).

The kindling intensity may vary depending on the nature of the protein, the time and wavelength of exposure, etc., but in certain embodiments ranges from about 1 to about 10³, from about 2 to about 5×10², from about 5 to about 100, or from about 10 to about 50 W/cm². In certain embodiments, the intensity ranges from about 10³ to about 10⁶, or from about 5×10³ to about 10⁵ W/cm² or more. As such, the intensity may range from about 1 to about 10⁵ W/cm², sometimes from about 2 to about 10⁶ W/cm², and sometimes from about 10 to about 50 W/cm².

After kindling, the kindling fluorescent protein is referred to as a “kindled” kindling fluorescent protein, where the kindled kindling fluorescent protein is fluorescent, i.e., it is in the fluorescent second state. Because the kindled kindling fluorescent proteins are fluorescent, they can be excited at a spectra of light wavelength(s), i.e., an excitation spectra, in a manner such that they emit light at a second spectra of wavelenghts, i.e., an emission spectra, where the second spectra is shifted, e.g., longer, than the excitation spectra.

The excitation spectra of the subject proteins varies depending on the particular protein, where in many embodiments the spectra typically have an excitation maximum that ranges from about 300 to about 700, from about 350 to about 650, or from about 400 to about 600 nm.

Likewise, the emission spectra of the subject kindled proteins varies depending on the particular protein, where in many embodiments the emission spectra typically have a maximum that ranges from about 400 to about 800, from about 425 to about 775, or from about 450 to about 750 nm. In many embodiments, the kindled proteins generally have a maximum extinction coefficient that ranges from about 5,000 to 50,000 and usually from about 15,000 to 45,000.

The fluorescent second state of the kindled proteins may be transient or permanent. Where the fluorescence of the kindled proteins is transient, the kindled protein may remain in the second fluorescent, i.e., kindled, state for a period of time from about 0.1 second to about 1 second, from about 1 second to about 60 seconds, from about 1 minute to about 10 minutes, from about 10 minutes to about minutes, from about 15 minutes to about 30 minutes, from about 30 minutes to about 45 minutes, from about 45 minutes to about 60 minutes, from about 1 hour to about 4 hours, from about 4 hours to about 12 hours, from about 12 hours to about 24 hours, from about 24 hours to about 36 hours, from about 36 hours to about 48 hours, from about 2 days to about 4 days, from about 4 days to about 7 days, from about 1 week to about 2 weeks, from about 2 weeks to about 1 month, from about 1 month to about 3 months, from about 3 months to about 6 months, from about 6 months to about 12 months, so that a protein's kindled state is considered transient if the fluorescent state is present for from about 0.1 seconds to 1 year following exposure to the kindling stimulus. Where the fluorescent state remains even after 1 year, e.g., from about 1 year to about 2 years, or longer, the kindled state is considered permanent and not transient. Where the fluorescence is transient, the rate at which fluorescence fades is constant in certain embodiments and variable in other embodiments.

In other embodiments, the kindled state is permanent, such that the kindled protein does not return to its first, non-kindled state.

In certain embodiments, the fluorescence of the kindled proteins can be quenched, i.e., removed, by exposure of the protein to a quenching wavelength of light, such that the protein is no longer fluorescent but returns to its first substantially non-fluorescent, if not non-fluorescent, state. Thus, in some embodiments, a kindling fluorescent protein of the invention can be turned “on” and “off” by exposure to selected wavelengths of light. For example, in some embodiments, a mutant KFP is kindled upon exposure to green light of a first intensity, fluoresces upon exposure to green light of a second, lower intensity, and fluorescence is quenched upon exposure to blue light. In general, quenching is reversible. Thus, it is possible to turn the fluorescence of a kindling protein on and off multiple times by exposure to different wavelengths of light. While varying depending on the particular protein, in many embodiments the quenching wavelength of light ranges from about 200 to about 1500, usually from about 300 to about 600, and in certain embodiments the quenching light is light of blue wavelength, ranging from about 300 to about 500.

In other embodiments, fluorescence of a kindled mutant KFP is not quenched at any wavelength, such that the kindled fluorescent protein is non-quenchable.

In certain embodiments, kindling fluorescent proteins of the invention are stable to exposure to a condition, including but not limited to, temperature extremes, desiccation, a denaturing agent, and in vivo conditions, as described in more detail below. By “stable” is meant that the protein retains at least about 25%, at least about 30%, at least about 40%, at least about 50%, at least about 60%, at least about 70%, at least about 80%, or at least about 90% or more, of at least one of the above-described kindling properties upon exposure to the condition for a period of time of at least about 5 minutes, at least about 15 minutes, at least about minutes, at least about 1 hour, at least about 2 hours, at least about 4 hours, at least about 12 hours, at least about 24 hours, at least about 2 days, at least about 1 week, at least about 2 weeks, at least about 1 month, at least about 2 months, at least about 6 months, at least about 1 year, or at least about 2 years, or longer. In some embodiments, the subject proteins are stable at temperatures in the range of from about 4° C. to about 40° C. In other embodiments, the subject proteins are stable at temperatures up to about 45° C., about 50° C., about 60° C., about 70° C., about 80° C., or about 90° C. In other embodiments, the subject proteins are stable at temperatures down to about 0° C., about −10° C., about −20° C., about −40° C., about −60° C., or about −70° C., or less.

In certain embodiments, the subject proteins are stable to desiccation, including air-drying and lyophilization. Thus, in some embodiments, when a protein of the invention is desiccated, then reconstituted, e.g., in a buffered solution, the reconstituted mutant KFP is stable. In some embodiments, when a protein of the invention is desiccated, it still preserves at least one of the above-described kindling properties.

In certain embodiments, a protein of the invention is stable in the presence of a denaturing agent (e.g., chaotropic agents, detergents, and the like), including, but not limited to guanidinium HCl (GuHCl) (e.g., up to 6M GuHCl); sodium dodecyl sulfate (SDS) (e.g., up to 1% SDS); and the like.

In certain embodiments of interest, a protein of the invention is stable in vivo in eukaryotic and/or prokaryotic cells.

In certain embodiments, a protein of the invention is storage stable. Thus, for example, a protein of the invention is stable to storage in an aqueous solution, as a lyophilized preparation, and the like, and remain stable for days, weeks, months, or usually years.

In certain embodiments, a protein of the invention is stable to changes in pH. Thus, for example, the protein of the invention is stable to a change in pH of 0.5, 1.0, 1.5, 2.0, 2.5, 3.0, 3.5, 4.0, 4.5, 5.0, 5.5, 6.0, or 6.5 pH units.

The subject kindling fluorescent proteins typically range in length from about 150 to 300 and usually from about 200 to 300 amino acid residues, and generally have a molecular weight ranging from about 15 to 35 kDa, usually from about 17.5 to 32.5 kDa.

The subject kindling fluorescent proteins may be naturally occurring proteins or non-naturally occurring proteins, e.g., mutant proteins of naturally occurring proteins. Natural sources of kindling fluorescent proteins include, but are not limited to, members of the phylum Cnidaria. Cnidarians include anthozoan species, scyphozoan species, and hydrozoan species. Specific naturally occurring sources of interest are kindling fluorescent proteins from the following specific anthozoan species: Anemonia majano, Clavularia sp., Zoanthus sp., Discosoma striata, Discosoma sp. “red”, Anemonia sulcata, Discosoma sp., Discosoma sp “green”, Discosoma sp.“magenta,” Heteractis sp., e.g., Heteractis crispa , and Condylactis sp., e.g., Condylactis gigantea. In certain embodiments, the proteins are further characterized in that they encode proteins that are either from: (1) non-bioluminescent species, often non-bioluminescent Cnidarian species, e.g., non-bioluminescent Anthozoan species; or (2) from Anthozoan species that are not Pennatulacean species, i.e., that are not sea pens; such that in these embodiments the proteins may be from bioluminescent Anthozoan species, so long as these species are not Pennatulacean species, e.g., that are not Renillan or Ptilosarcan species. In certain embodiments, the kindling proteins of interest are non-naturally occurring proteins, as non-naturally occurring proteins exhibit enhanced kindling properties, e.g., enhanced fluorescence lifetime, brighter fluorescence, etc., as compared to naturally occurring proteins that are kindling proteins. For example, in some embodiments, a mutant protein of the invention remains fluorescent (i.e., in the kindled state) for a period of time that is at least about 25%, at least about 30%, at least about 40%, at least about 50%, at least about 75%, at least about 100% (or two-fold), at least about 5-fold, at least about 10-fold, at least about 20-fold, at least about 50-fold more, or longer, when compared to a reference protein, e.g., a non-mutated form of the protein. In other embodiments, the kindling fluorescent proteins of the subject invention are naturally occurring proteins that do not include the wild type asFP595 (also known as asCP), as described below.

### A. sulcata

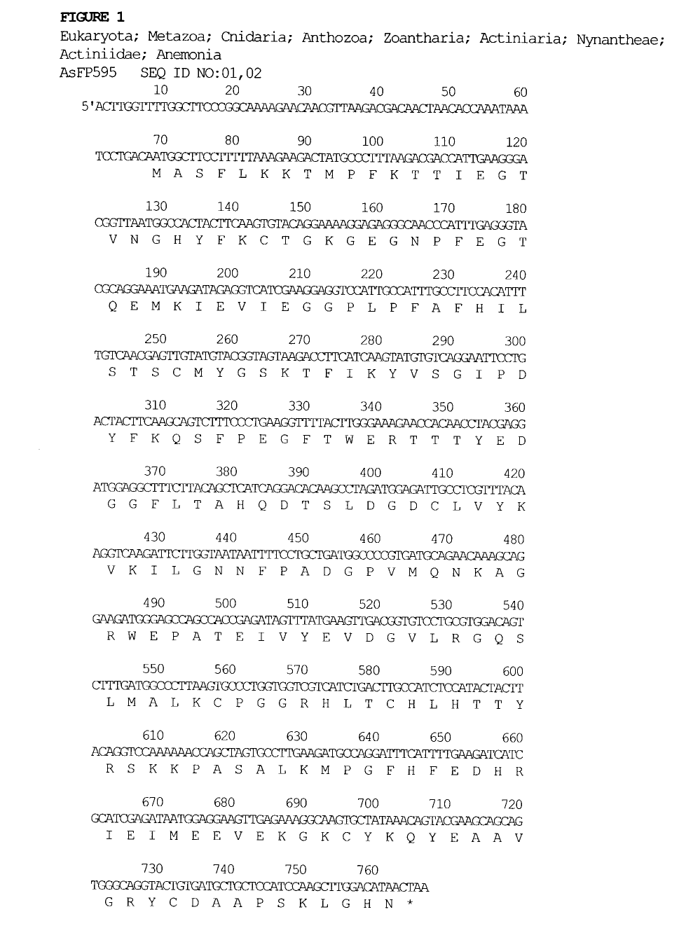

Of particular interest in certain embodiments is the asFP595(asFP600) (NFP-7) (asCP) protein that has an amino acid sequence as shown in FIG. 1 and identified as SEQ ID NO:02. AsFP595 is further described in U.S. patent application Ser. No. 10/006,922; the disclosure of which is incorporated by reference herein. In other embodiments, the kindling proteins are mutants of this wild type protein, where particular mutants of interest include, but are not limited to: (a) KFP1, (KFP 04a) A148G; (b) KFP 04b (F90L; A148G; H203Y); and the like; as described in greater detail below.

### Heteractis crispa

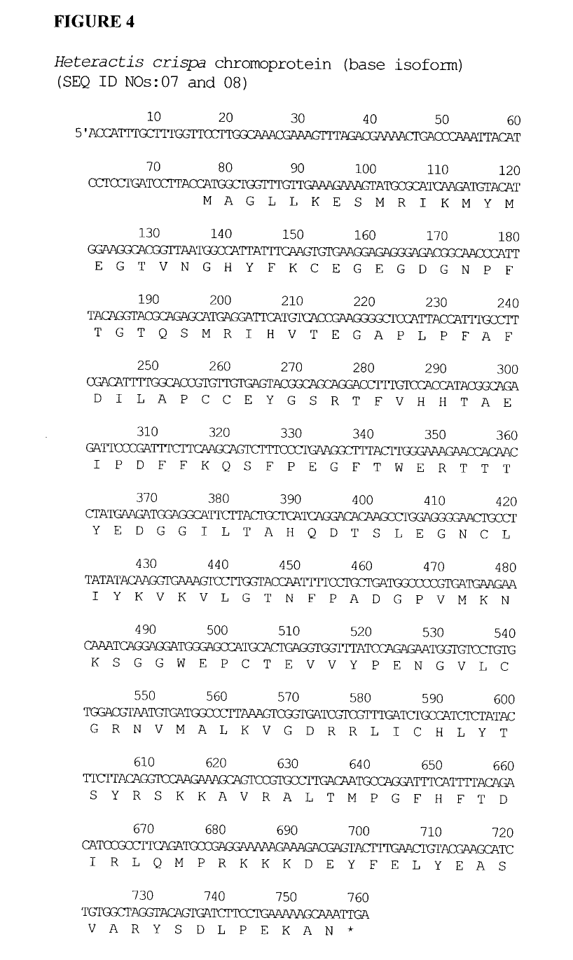

In other particular embodiments, of particular interest is the protein known as Heteractis crispa chromoprotein, or mutants thereof. H. crispa chromoprotein has an amino acid sequence depicted in FIG. 4 and identified as SEQ ID NO:08. Examples of mutants of H. crispa that are KFP are those having amino acid sequence set forth in SEQ ID NOs:10, 12,14, and 16; as described in greater detail below.

Proteins of interest also include proteins that differ in amino acid sequence by at least one amino acid from the specific sequences provided above. Homologs or proteins (or fragments thereof) that vary in sequence from the above provided specific sequences are also provided. By homolog is meant a protein having at least about 10%, usually at least about 20 % and more usually at least about 30 %, and in many embodiments at least about 35 %, usually at least about 40% and more usually at least about 60 %, at least about 70%, at least about 80%, at least about 90%, at least about 95%, at least about 97%, at least about 98%, or at least about 99% amino acid sequence identity to one of the specific amino acid sequences provided herein, as determined using MegAlign, DNAstar (1998) clustal algorithm as described in D. G. Higgins and P. M. Sharp, “Fast and Sensitive multiple Sequence Alignments on a Microcomputer,” (1989) CABIOS, 5: 151-153. (Parameters used are ktuple 1, gap penalty 3, window, 5 and diagonals saved 5). In many embodiments, homologues of interest have much higher sequence identity, e.g., 65%, 70%, 75%, 80%, 85%, 90% or higher (e.g., 98%, 99%, 99.5%, 99.8%, 99.9%).

In certain embodiments of interest, a non-naturally occurring kindling fluorescent protein of the invention includes amino acid substitutions at one or more of positions 148, 165, 203 as determined using the GFP alignment protocol as disclosed in Matz et al., infra. In particular embodiments of interest, a protein of the invention includes one or more of the following amino acid changes when compared to SEQ ID NO:02 as described above: (1) A148G; and (2) H203Y. Those skilled in the art will recognize that the specific amino acid positions will vary from protein to protein. Those skilled in the art can readily determine, based on these guidelines, which amino acid corresponds to 148, 165, and 203 in any given chromo- or fluoroprotein.

In further embodiments of interest, a protein of the invention includes amino acid substitutions at one or more of positions K28, N165, G20, T39, L126, C148, L126, R176, L181, A190, 1203, P208, and K211, when compared to SEQ ID NO:08. In particular embodiments of interest, a protein of the invention includes one or more of the following amino acid changes when compared to SEQ ID NO:08: (1) K28M; (2) N165A; (3) N165G; (4) G20C; (5) T39A; (6) L126H; (7) C148A; (8) RI76H; (9) L181H; (10) A190V; (11) 1203H; (12) P208L; (13) K211E; (14) C148S; (15) N165S; and (16) P208R. Those skilled in the art will recognize that the specific amino acid positions will vary from protein to protein. Those skilled in the art can readily determine, based on these guidelines, which amino acid corresponds to the aforementioned altered amino acids in any given chromo- or fluoroprotein.

The subject proteins are present in a non-naturally occurring environment. In certain embodiments, the subject proteins are provided as purified proteins, where by purified is meant that the protein is present in a composition that is substantially free of proteins other than a subject protein, where by substantially free is meant that less than 90%, usually less than 60% and more usually less than 50% of the composition is made up of proteins other than a subject kindling fluorescent protein.

The proteins of the subject invention may also be present as an isolate, by which is meant that the protein is substantially free of other proteins and other naturally occurring biologic molecules, such as oligosaccharides, polynucleotides and fragments thereof, and the like, where the term “substantially free” in this instance means that less than 70%, usually less than 60% and more usually less than 50% of the composition containing the isolated protein is some other naturally occurring biological molecule. In certain embodiments, the proteins are present in substantially pure form, where by “substantially pure form” is meant at least 95%, usually at least 97% and more usually at least 99% pure.

For those proteins of the subject invention that are naturally occurring proteins, the proteins are present in a non-naturally occurring environment, e.g., are separated from their naturally occurring environment. In certain embodiments, the subject proteins are present in a composition that is enriched for the subject protein as compared to its naturally occurring environment. For example, purified protein is provided, where by purified is meant that the protein is present in a composition that is substantially free of non-kindling proteins of interest, where by substantially free is meant that less than 90%, usually less than 60% and more usually less than 50% of the composition is made up of non-kindling proteins of interest. The proteins of the subject invention may also be present as an isolate, by which is meant that the protein is substantially free of other proteins and other naturally occurring biologic molecules, such as oligosaccharides, polynucleotides and fragments thereof, and the like, where the term “substantially free” in this instance means that less than 70%, usually less than 60% and more usually less than 50% of the composition containing the isolated protein is some other naturally occurring biological molecule. In certain embodiments, the proteins are present in substantially pure form, where by “substantially pure form” is meant at least 95%, usually at least 97% and more usually at least 99% pure.

Fragments of the subject proteins are also provided. Generally such polypeptides include an amino acid sequence encoded by an open reading frame (ORF) of the gene encoding a kindling fluorescent protein, including the full length protein and fragments thereof, particularly biologically active fragments and/or fragments corresponding to functional domains; and including fusions of the subject polypeptides to other proteins or parts thereof. Fragments of interest will typically be at least about 10 amino acids (aa) in length, usually at least about 50 aa in length, and may be as long as 300 aa in length or longer, but will usually not exceed about 1000 aa in length, where the fragment will have a stretch of amino acids that is identical to the subject protein of at least about 10 aa, and usually at least about 15 aa, and in many embodiments at least about 50 aa in length. In some embodiments, the subject polypeptides are about 25 aa, about 50 aa, about 75 aa, about 100 aa, about 125 aa, about 150 aa, about 200 aa, about 210 aa, about 220 aa, about 230 aa, or about 240 aa in length, up to the entire protein. In some embodiments, a protein fragment retains all or substantially all of a biological property of the parent protein.

The subject proteins are in many embodiments synthetically or recombinantly produced. For example, the subject proteins may be derived by recombinant means, e.g. by expressing a recombinant gene or nucleic acid coding sequence encoding the protein of interest in a suitable host, as described above. Any convenient protein purification procedures may be employed, where suitable protein purification methodologies are described in Guide to Protein Purification, (Deuthser ed.) (Academic Press, 1990). For example, a lysate may be prepared from the original source and purified using HPLC, exclusion chromatography, gel electrophoresis, affinity chromatography, and the like.

### Nucleic Acid Compositions

As summarized above, the subject invention also provides nucleic acid compositions encoding the above described proteins, as well as fragments and homologues of these proteins. By nucleic acid composition is meant a composition comprising a sequence of DNA having an open reading frame that encodes a protein of the subject invention and is capable, under appropriate conditions, of being expressed as a protein according to the subject invention. Also encompassed in this term are nucleic acids that are homologous, substantially similar or identical to the nucleic acids of the present invention. Thus, the subject invention provides genes and coding sequences thereof encoding the proteins of the subject invention, as well as homologs thereof. Where the subject nucleic acids are naturally occurring, they are present in other than their natural environment, e.g., they are isolated, present in enriched amounts, etc., from their naturally occurring environment, e.g., the organism from which they are obtained.

Specific nucleic acids of interest include those provided herein as SEQ ID NOs: 01, 03, 05; 07, 09 and 11.

In addition to the above-described specific nucleic acid compositions, also of interest are homologues of the above sequences. With respect to homologues of the subject nucleic acids, the source of homologous genes may be any species of plant or animal or the sequence may be wholly or partially synthetic. In certain embodiments, sequence similarity between homologues is at least about 20%, sometimes at least about 25%, and may be 30%, 35%, 40%, 50%, 60%, 70% or higher, including 75%, 80%, 85%, 90% and 95% or higher. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17). The sequences provided herein are essential for recognizing related and homologous nucleic acids in database searches. Of particular interest in certain embodiments are nucleic acids of substantially the same length as the nucleic acid identified as SEQ ID NOS: 01, 03, 05, 07, 09 and 11, where by substantially the same length is meant that any difference in length does not exceed about 20 number %, usually does not exceed about 10 number % and more usually does not exceed about 5 number %; and have sequence identity to any of these sequences of at least about 90%, usually at least about 95% and more usually at least about 99% over the entire length of the nucleic acid. In many embodiments, the nucleic acids have a sequence that is substantially similar (i.e. the same as) or identical to the sequences of SEQ ID NOS: 01, 03, 05 and 07. By substantially similar is meant that sequence identity will generally be at least about 60%, usually at least about 75% and often at least about 80, 85, 90, or even 95%.

Also provided are nucleic acids that encode the proteins encoded by the above described nucleic acids, but differ in sequence from the above described nucleic acids due to the degeneracy of the genetic code.

Also provided are nucleic acids that hybridize to the above-described nucleic acid under stringent conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

Nucleic acids encoding variants, e.g., mutants, of the above specifically provided proteins of the invention are also provided. Such nucleic acids can be generated by random mutagenesis or targeted mutagenesis, using well-known techniques which are routine in the art. In some embodiments, proteins encoded by nucleic acids encoding homologues or mutants have the same properties as the parent protein, while in other embodiments, the encoded product varies from the parent protein in some aspect.

In certain embodiments, the protein that is encoded by the nucleic acid is a non-aggregating protein. In many embodiments, the non-aggregating proteins differ from a parent sequence by an alternation in the N-terminus that modulates the charges appearing on side groups of the N-terminus residues, e.g., to reverse or neutralize the charge, in a manner sufficient to produce a non-aggregating variant of parent sequence, where a particular protein is considered to be non-aggregating if it is determined be non-aggregating using the assay reported in U.S. Patent Application serial No. 60/270,983, the disclosure of which is herein incorporated by reference. More specifically, basic residues located near the N-termini of the proteins are substituted, e.g., Lys and Arg residues close to the N-terminus are substituted with negatively charged or neutral residues.

Another category of variant of particular interest is the modulated oligomerization variant. A variant is considered to be a modulated oligomerization variant if its oligomerization properties are different as compared to a reference or parent protein, e.g., a wild type protein. For example, if a particular variant oligomerizes to a greater or lesser extent than the wild type, it is considered to be an oligomerization variant. Of particular interest are oligomerization variants that do not oligomerize, i.e., are monomers under physiological (e.g., intracellular) conditions, or oligomerize to a lesser extent that the wild type, e.g., are dimers or trimers under intracellular conditions, e.g., in a controlled manner.

Nucleic acids of the subject invention may be cDNA or genomic DNA or a fragment thereof. In certain embodiments, the nucleic acids of the subject invention include one or more of the open reading frames encoding specific kindling fluorescent proteins and polypeptides, and introns, as well as adjacent 5′ and 3′ non-coding nucleotide sequences involved in the regulation of expression, up to about 20 kb beyond the coding region, but possibly further in either direction. The subject nucleic acids may be introduced into an appropriate vector for extrachromosomal maintenance or for integration into a host genome, as described in greater detail below.

The term “cDNA” as used herein is intended to include all nucleic acids that share the arrangement of sequence elements found in native mature mRNA species, where sequence elements are exons and 5′ and 3′ non-coding regions. Normally mRNA species have contiguous exons, with the intervening introns, when present, being removed by nuclear RNA splicing, to create a continuous open reading frame encoding the protein.

A genomic sequence of interest comprises the nucleic acid present between the initiation codon and the stop codon, as defined in the listed sequences, including all of the introns that are normally present in a native chromosome. It may further include 5′ and 3′ un-translated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

The nucleic acid compositions of the subject invention may encode all or a part of the subject proteins. Double or single stranded fragments may be obtained from the DNA sequence by chemically synthesizing oligonucleotides in accordance with conventional methods, by restriction enzyme digestion, by PCR amplification, etc. For the most part, DNA fragments will be of at least about 15 nt, usually at least about 18 nt or about 25 nt, and may be at least about 50 nt. In some embodiments, the subject nucleic acid molecules may be about 100 nt, about 200 nt, about 300 nt, about 400 nt, about 500 nt, about 600 nt, about 700 nt, or about 720 nt in length. The subject nucleic acids may encode fragments of the subject proteins or the full-length proteins, e.g., the subject nucleic acids may encode polypeptides of about 25 aa, about 50 aa, about 75 aa, about 100 aa, about 125 aa, about 150 aa, about 200 aa, about 210 aa, about 220 aa, about 230 aa, or about 240 aa, up to the entire protein.

The subject nucleic acids are isolated and obtained in substantial purity, generally as other than an intact chromosome. Usually, the DNA will be obtained substantially free of other nucleic acid sequences that do not include a nucleic acid of the subject invention or fragment thereof, generally being at least about 50%, usually at least about 90% pure and are typically “recombinant”, i.e. flanked by one or more nucleotides with which it is not normally associated on a naturally. occurring chromosome.

The subject polynucleotides (e.g., a polynucleotide having a sequence of SEQ ID NOS: 01; 03, 05 and 07; etc.), the corresponding cDNA, the full-length gene and constructs of the subject polynucleotides are provided. These molecules can be generated synthetically by a number of different protocols known to those of skill in the art. Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y., and under current regulations described in United States Dept. of HHS, National Institute of Health (NIH) Guidelines for Recombinant DNA Research.

Also provided are nucleic acids that encode fusion proteins of the subject proteins, or fragments thereof, which are fused to a second protein, e.g., a degradation sequence, a signal peptide, a protein of interest to be studied in a method where the subject kindling fluorescent protein is employed as a photoactivatable marker, etc. Fusion proteins may comprise a subject polypeptide, or fragment thereof, and a non-kindling fluorescent protein polypeptide (“the fusion partner” ) fused in-frame at the N-terminus and/or C-terminus of the subject polypeptide. Fusion partners include, but are not limited to, polypeptides that can bind antibody specific to the fusion partner (e.g., epitope tags); antibodies or binding fragments thereof; polypeptides that provide a catalytic function or induce a cellular response; ligands or receptors or mimetics thereof; proteins of interest to be studied, and the like. In such fusion proteins, the fusion partner is generally not naturally associated with the subject kindling fluorescent protein portion of the fusion protein, and in certain embodiments is not an Anthozoan protein or derivative/fragment thereof, i.e., it is not found in Anthozoan species.

Also provided are constructs comprising the subject nucleic acids inserted into a vector, where such constructs may be used for a number of different applications, including propagation, protein production, etc. Viral and non-viral vectors may be prepared and used, including plasmids. The choice of vector will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially.

In certain embodiments, the vector is a member of a recombinase based system in which a recombinase mediates the transfer of a nucleic acid from a donor to an acceptor vector, where the donor and acceptor vectors each include at least one recombinase recognition site. A variety of different site-specific recombinase systems suitable for transferring a nucleic acid from a donor to an acceptor vector are known and may be modified to be useful in the subject invention. Such systems include those described in U.S. Pat. Nos. 5,851,808; 5,888,732; and U.S. Provisional application Ser. No. 09/616,651, the disclosure of which are herein incorporated by reference, as well as WO 00/12687 and WO 01/05961, the disclosures of the priority documents of which are herein incorporated by reference.

To prepare the constructs, the partial or full-length polynucleotide is inserted into a vector typically by means of DNA ligase attachment to a cleaved restriction enzyme site in the vector. Alternatively, the desired nucleotide sequence can be inserted by homologous recombination in vivo. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

Also provided are expression cassettes or systems that find use in, among other applications, the synthesis of the subject proteins. For expression, the gene product encoded by a polynucleotide of the invention is expressed in any convenient expression system, including, for example, bacterial, yeast, insect, amphibian and mammalian systems. Suitable vectors and host cells are described in U.S. Pat. No. 5,654,173. In the expression vector, a subject polynucleotide, e.g., as set forth in SEQ ID NOS:01; 03; 05; 07; 09; 11; 13; 15 or 17, is linked to a regulatory sequence as appropriate to obtain the desired expression properties. These regulatory sequences can include promoters (attached either at the 5′ end of the sense strand or at the 3′ end of the antisense strand), enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used. In other words, the expression vector will provide a transcriptional and translational initiation region, which may be inducible or constitutive, where the coding region is operably linked under the transcriptional control of the transcriptional initiation region, and a transcriptional and translational termination region. These control regions may be native to the subject species from which the subject nucleic acid is obtained, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for, among other things, the production of fusion proteins, as described above.

Expression cassettes may be prepared comprising a transcription initiation region, the gene or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, and up to the complete open reading frame of the gene. After introduction of the DNA, the cells containing the construct may be selected by means of a selectable marker, the cells expanded and then used for expression.

The above described expression systems may be employed with prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, e.g. COS 7 cells, HEK 293, CHO, Xenopus Oocytes, etc., may be used as the expression host cells. In some situations, it is desirable to express the gene in eukaryotic cells, where the expressed protein will benefit from native folding and post-translational modifications. Small peptides can also be synthesized in the laboratory. Polypeptides that are subsets of the complete protein sequence may be used to identify and investigate parts of the protein important for function.

Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. RE 30,985.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to a selected polynucleotide is identified, its expression can be regulated in the cell to which the gene is native. For example, an endogenous gene of a cell can be regulated by an exogenous regulatory sequence inserted into the genome of the cell at location sufficient to at least enhance expressed of the gene in the cell. The regulatory sequence may be designed to integrate into the genome via homologous recombination, as disclosed in U.S. Pat. Nos. 5,641,670 and 5,733,761, the disclosures of which are herein incorporated by reference, or may be designed to integrate into the genome via non-homologous recombination, as described in WO 99/15650, the disclosure of which is herein incorporated by reference. As such, also encompassed in the subject invention is the production of the subject proteins without manipulation of the encoding nucleic acid itself, but instead through integration of a regulatory sequence into the genome of cell that already includes a gene encoding the desired protein, as described in the above incorporated patent documents.

Also provided are homologs of the subject nucleic acids. Homologs are identified by any of a number of methods. A fragment of the provided cDNA may be used as a hybridization probe against a cDNA library from the target organism of interest, where low stringency conditions are used. The probe may be a large fragment, or one or more short degenerate primers. Nucleic acids having sequence similarity are detected by hybridization under low stringency conditions, for example, at 50° C. and 6×SSC (0.9 M sodium chloride/0.09 M sodium citrate) and remain bound when subjected to washing at 55° C. in 1×SSC (0.15 M sodium chloride/.015 M sodium citrate). Sequence identity may be determined by hybridization under stringent conditions, for example, at 50° C. or higher and 0.1×SSC (15 mM sodium chloride/1.5 mM sodium citrate). Nucleic acids having a region of substantial identity to the provided sequences, e.g. allelic variants, genetically altered versions of the gene, etc., bind to the provided sequences under stringent hybridization conditions. By using probes, particularly labeled probes of DNA sequences, one can isolate homologous or related genes.

Also of interest are promoter elements of the subject genomic sequences, where the sequence of the 5′ flanking region may be utilized for promoter elements, including enhancer binding sites, e.g., that provide for regulation of expression in cells/tissues where the subject proteins gene are expressed.

Also provided are small DNA fragments of the subject nucleic acids, which fragments are useful as primers for PCR, hybridization screening probes, etc. Larger DNA fragments, i.e., greater than 100 nt are useful for production of the encoded polypeptide, as described in the previous section. For use in geometric amplification reactions, such as geometric PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

The DNA may also be used to identify expression of the gene in a biological specimen. The manner in which one probes cells for the presence of particular nucleotide sequences, as genomic DNA or RNA, is well established in the literature. Briefly, DNA or mRNA is isolated from a cell sample. The mRNA may be amplified by RT-PCR, using reverse transcriptase to form a complementary DNA strand, followed by polymerase chain reaction amplification using primers specific for the subject DNA sequences. Alternatively, the mRNA sample is separated by gel electrophoresis, transferred to a suitable support, e.g. nitrocellulose, nylon, etc., and then probed with a fragment of the subject DNA as a probe. Other techniques, such as oligonucleotide ligation assays, in situ hybridizations, and hybridization to DNA probes arrayed on a solid chip may also find use. Detection of mRNA hybridizing to the subject sequence is indicative of kindling fluorescent protein gene expression in the sample.

The subject nucleic acids, including flanking promoter regions and coding regions, may be mutated in various ways known in the art to generate targeted changes in promoter strength, sequence of the encoded protein, properties of the encoded protein, including fluorescent properties of the encoded protein, etc. The DNA sequence or protein product of such a mutation will usually be substantially similar to the sequences,provided herein, e.g. will differ by at least one nucleotide or amino acid, respectively, and may differ by at least two but not more than about ten nucleotides or amino acids. The sequence changes may be substitutions, insertions, deletions, or a combination thereof. Deletions may further include larger changes, such as deletions of a domain or exon, e.g. of stretches of 10, 20, 50, 75, 100, 150 or more aa residues. Techniques for in vitro mutagenesis of cloned genes are known. Examples of protocols for site specific mutagenesis may be found in Gustin et al. (1993), Biotechniques 14:22; Barany (1985), Gene 37:111-23; Colicelli et al. (1985), Mol. Gen. Genet. 199:537-9; and Prentki et al. (1984), Gene 29:303-13. Methods for site specific mutagenesis can be found in Sambrook et al., Molecular Cloning: A Laboratory Manual, CSH Press 1989, pp. 15.3-15.108; Weiner et al. (1993), Gene 126:35-41; Sayers et al. (1992), Biotechniques 13:592-6; Jones and Winistorfer (1992), Biotechniques 12:528-30; Barton et al. (1990), Nucleic Acids Res 18:7349-55; Marotti and Tomich (1989), Gene Anal. Tech. 6:67-70; and Zhu (1989), Anal Biochem 177:120-4. Such mutated nucleic acid derivatives may be used to study structure-function relationships of a particular chromo/fluorescent protein, or to alter properties of the protein that affect its function or regulation.

Also of interest are humanized versions of the subject nucleic acids. As used herein, the term “humanized” refers to changes made to the a nucleic acid sequence to optimize the codons for expression of the protein in human cells (Yang et al., Nucleic Acids Research 24 (1996), 4592-4593). See also U.S. Pat. No. 5,795,737 which describes humanization of proteins, the disclosure of which is herein incorporated by reference.

### Antibody Compositions

Also provided are antibodies that specifically bind to the subject kindling fluorescent proteins. In many embodiments, a subject antibody is isolated, e.g., is in an environment other than its naturally-occurring environment. Suitable antibodies are obtained by immunizing a host animal with peptides comprising all or a portion of the subject protein. Suitable host animals include mouse, rat sheep, goat, hamster, rabbit, etc. The host animal will generally be from a different species than the immunogen where the immunogen is from a naturally occurring source, e.g., an anthozoan species, where representative host animals include, but are not limited to, e.g., rabbits, goats, mice, etc.

The immunogen may comprise the complete protein, or fragments and derivatives thereof. Preferred immunogens comprise all or a part of the protein, where these residues contain the post-translation modifications found on the native target protein. Immunogens are produced in a variety of ways known in the art, e.g., expression of cloned genes using conventional recombinant methods, preparation of fragments of a subject mutant protein using well-known methods, etc.

For preparation of polyclonal antibodies, the first step is immunization of the host animal with the target protein, where the target protein will preferably be in substantially pure form, comprising less than about 1% contaminant. The immunogen may comprise the complete target protein, fragments or derivatives thereof. To increase the immune response of the host animal, the target protein may be combined with an adjuvant, where suitable adjuvants include alum, dextran, sulfate, large polymeric anions, oil & water emulsions, e.g. Freund's adjuvant, Freund's complete adjuvant, and the like. The target protein may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The target protein is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using protein bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Also provided are “artificial” antibodies, e.g., antibodies and antibody fragments produced and selected in vitro. In some embodiments, such antibodies are displayed on the surface of a bacteriophage or other viral particle. In many embodiments, such artificial antibodies are present as fusion proteins with a viral or bacteriophage structural protein, including, but not limited to, M13 gene III protein. Methods of producing such artificial antibodies are well known in the art. See, e.g., U.S. Pat. Nos. 5,516,637; 5,223,409; 5,658,727; 5,667,988; 5,498,538; 5,403,484; 5,571,698; and 5,625,033.

Also of interest in certain embodiments are humanized antibodies. Methods of humanizing antibodies are known in the art. The humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) Proc. Natl. Acad. Sci. USA. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods. Other methods for preparing chimeric antibodies are described in, e.g., U.S. Pat. No. 5,565,332.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions may be used to,design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et al. (1985) Cell 41:885); native Ig promoters, etc.

### Transgenics

The subject nucleic acids can be used to generate transgenic, non-human plants or animals or site specific gene modifications in cell lines. Transgenic cells of the subject invention include one or more nucleic acids according to the subject invention present as a transgene, where included within this definition are the parent cells transformed to include the transgene and the progeny thereof. In many embodiments, the transgenic cells are cells that do not normally harbor or contain a nucleic acid according to the subject invention. In those embodiments where the transgenic cells do naturally contain the subject nucleic acids, the nucleic acid will be present in the cell in a position other than its natural location, i.e., integrated into the genomic material of the cell at a non-natural location. Transgenic animals may be made through homologous recombination, where the endogenous locus is altered. Alternatively, a nucleic acid construct is randomly integrated into the genome. Vectors for stable integration include plasmids, retroviruses and other animal viruses, HACs, YACs, and the like.

Transgenic organisms of the subject invention include cells and multicellular organisms, e.g., plants and animals, that are endogenous knockouts in which expression of the endogenous gene is at least reduced if not eliminated. Transgenic organisms of interest also include cells and multicellular organisms, e.g., plants and animals, in which the protein or variants thereof is expressed in cells or tissues where it is not normally expressed and/or at levels not normally present in such cells or tissues.

DNA constructs for homologous recombination will comprise at least a portion of the gene of the subject invention, wherein the gene has the desired genetic modification(s), and includes regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990), Meth. Enzymol. 185:527-537.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture. The transgenic animals may be any non-human mammal, such as laboratory animals, domestic animals, etc. The transgenic animals may be used in functional studies, drug screening, etc. Representative examples of the use of transgenic animals include those described infra. Transgenic plants may be produced in a similar manner. Methods of preparing transgenic plant cells and plants are described in U.S. Pat. Nos. 5,767,367; 5,750,870; 5,739,409; 5,689,049; 5,689,045; 5,674,731; 5,656,466; 5,633,155; 5,629,470; 5,595,896; 5,576,198; 5,538,879; 5,484,956; the disclosures of which are herein incorporated by reference. Methods of producing transgenic plants are also reviewed in Plant Biochemistry and Molecular Biology (eds Lea & Leegood, John Wiley & Sons)(1993) pp 275-295. In brief, a suitable plant cell or tissue is harvested, depending on the nature of the plant species. As such, in certain instances, protoplasts will be isolated, where such protoplasts may be isolated from a variety of different plant tissues, e.g. leaf, hypoctyl, root, etc.

For protoplast isolation, the harvested cells are incubated in the presence of cellulases in order to remove the cell wall, where the exact incubation conditions vary depending on the type of plant and/or tissue from which the cell is derived. The resultant protoplasts are then separated from the resultant cellular debris by sieving and centrifugation. Instead of using protoplasts, embryogenic explants comprising somatic cells may be used for preparation of the transgenic host. Following cell or tissue harvesting, exogenous DNA of interest is introduced into the plant cells, where a variety of different techniques are available for such introduction.

With isolated protoplasts, the opportunity arise for introduction via DNA-mediated gene transfer protocols, including: incubation of the protoplasts with naked DNA, e.g. plasmids, comprising the exogenous coding sequence of interest in the presence of polyvalent cations, e.g. PEG or PLO; and electroporation of the protoplasts in the presence of naked DNA comprising the exogenous sequence of interest. Protoplasts that have successfully taken up the exogenous DNA are then selected, grown into a callus, and ultimately into a transgenic plant through contact with the appropriate amounts and ratios of stimulatory factors, e.g. auxins and cytokinins. With embryogenic explants, a convenient method of introducing the exogenous DNA in the target somatic cells is through the use of particle acceleration or “gene-gun” protocols.

The resultant explants are then allowed to grow into chimera plants, cross-bred and transgenic progeny are obtained. Instead of the naked DNA approaches described above, another convenient method of producing transgenic plants is Agrobacterium mediated transformation. With Agrobacterium mediated transformation, co-integrative or binary vectors comprising the exogenous DNA are prepared and then introduced into an appropriate Agrobacterium strain, e.g. A. tumefaciens. The resultant bacteria are then incubated with prepared protoplasts or tissue explants, e.g. leaf disks, and a callus is produced. The callus is then grown under selective conditions, selected and subjected to growth media to induce root and shoot growth to ultimately produce a transgenic plant.

### Compositions

The present invention further provides compositions comprising the polypeptides, polynucleotides, recombinant vectors, host cells, and antibodies of the invention. These compositions may include a buffer, which is selected according to the desired use of the polypeptide, polynucleotide, recombinant vector, host cell, or antibody, and may also include other substances appropriate to the intended use. Those skilled in the art can readily select an appropriate buffer, a wide variety of which are known in the art, suitable for an intended use.

### Utility

The above described protein and nucleic acid compositions find use as biological labels or markers in a variety of different types of applications. Applications in which the subject protein or nucleic acid compositions find use are, in many embodiments, generally described as methods of detecting an entity in a composition, i.e., assaying a composition for the presence of an entity, by performing the following steps: (a) providing the entity to be detected as a labeled entity that is labeled with a kindling fluorescent protein of the present invention; (b) kindling the kindling fluorescent protein label to produce a kindled kindling fluorescent protein; and exciting the kindled protein and detecting any resultant fluorescence to detect the entity of interest in the composition being assayed. Each of these steps is now described separately in greater detail.

The composition that is assayed for the presence of the entity may vary depending on the particular application being performed. As such, the composition may be an intracellular composition, e.g., where detection of an intracellular protein, organelle, etc., is of interest; or a composition that includes one or more cells, e.g., where a particular type of cell in a complex mixture of cells is to be detected, etc.

The entity to be detected may be a variety of physiological entities, e.g., proteins, non-protein macromolecules, organelles, cells, organisms, etc.

As mentioned above, the first step in the subject methods is generally to provide the entity to be detected as an entity labeled with a kindling fluorescent protein, as described above. Depending on the nature of the entity, the entity may be labeled with the kindling fluorescent protein using a number of different protocols. For example, where the entity is a protein, the entity may be present in the mixture as a fusion protein with a kindling fluorescent protein, as described above, where construction and expression of vectors encoding fusion proteins is well known to those of skill in the art. Alternatively, a specific binding pair member that specifically binds to the entity of interest, e.g., an antibody or affinity reagent, mimetic thereof, e.g., as described above, may be employed, where the affinity reagent is labeled with a kindling fluorescent protein according to the present invention. In these latter embodiments, the to be detected entity is provided as a kindling fluorescent protein labeled entity by contacting the entity with the kindling fluorescent protein labeled affinity reagent under conditions sufficient for the affinity reagent to specifically bind the entity of interest. Any convenient manner of providing the entity to be detected as an entity labeled with the kindling fluorescent protein may be employed, the above two protocols being merely representative of the different approaches that may be used.

Following provision of the kindling fluorescent protein labeled entity, as described above, the label is subjected to a kindling stimulus in order to convert the label to the second, fluorescent state, i.e., the detectable kindled state. As reviewed above, the kindling stimulus is light of a sufficient wavelength, intensity and duration to produce change the protein from the first to the second state. In other words, the kindling stimulus is light of kindling wavelength, kindling intensity and kindling duration to change the protein from the first to the second state.

The kindling wavelength of light varies depending on the particular kindling proteins, and includes, but is not limited to, wavelengths in the visible spectrum, e.g., red, orange, yellow, green, blue, indigo, and violet; ultraviolet light; and infrared light. Thus, in many embodiments, a kindling wavelength of light is in the range of from about 190 nm to about 200 nm, from about 200 nm to about 300 nm, from about 300 nm to about 400 nm, from about 380 nm to about 400 nm, from about 400 nm to about 430 nm, from about 430 nm to about 500 nm, from about 500 nm to about 560 nm, from about 560 nm to about 620 nm, from about 620 nm. to about 700 nm, from about 700 nm to about 1.5 μm, from about 1.5 μm to about 20 μm, or from about 20μm to about 1000 μm. Additional ranges are provided above. The kindling wavelength may be a single wavelength or two or more different wavelengths.

The kindling fluorescent protein is exposed to the kindling wavelength for a kindling duration, i.e., discrete period of time, e.g., from about 0.001 second to about 0.005 second, from about 0.005 second to about 0.01 second, from about 0.01 second to about 0.05 second, from about 0.05 second to about 0.1 second, from about 0.1 second to about 0.5 second, from about 0.5 second to about 1 second, from about 1 second to about 5 seconds, from about 5 seconds to about 10 seconds, from about 10 seconds to about 15 seconds, from about 15 seconds to about 30 seconds, or from about 30 seconds to about 60 seconds, from about 1 minute to about 5 minutes, or from about 5 minutes to about 10 minutes, or longer. Additional ranges are provided above. The length of time to which the protein is exposed to a kindling wavelength of light depends, in part, on the intensity of the kindling light. Thus, in general, the higher the intensity of light, the shorter the time of exposure that is required to kindle the protein (i.e., to induce the conformation change that allows the protein to fluoresce).

The kindling intensity may vary depending on the nature of the protein, the time and wavelength of exposure, etc., but in certain embodiments ranges from about 1 to about 10³, from about 2 to about 5×10², from about 5 to about 100, or from about 10 to about 50 W/cm². In certain embodiments, the intensity ranges from about 10³ to about 10⁶, or from about 5×10³ to about 10⁵ W/cm² or more. Additional ranges are provided above.

The source of the kindling stimulus as described above may be any convenient source, e.g., a laser, a fluorescent microscope, a confocal microscope, or other light source capable of generating the requisite kindling stimulus,.where such sources are well known to those of skill in the art.

Following kindling of the label, as described above, the label is excited at a suitable excitation wavelength and any resultant fluorescence is detected to detect the presence of the entity in the assayed mixture. The excitation wavelength may be any convenient wavelength capable of exciting the fluorescent kindled protein label, where ranges for excitation are provided above. The source of the light can be a laser, a fluorescent microscope, a confocal microscope, or other light source that produces the desired wavelength and intensity.

After exposure of the label to the excitation wavelength, resultant fluorescence is detected. In many embodiments, “time zero” is the time at which exposure of the kindled protein to the excitation wavelength of light ends. In these embodiments, fluorescence is observed and/or measured beginning at time zero, or any time thereafter. In some embodiments, exposure to the kindling wavelength of light and the excitation wavelength of light is performed simultaneously.

Resultant detected fluorescence is then employed to detect the entity of interest by relating the presence of the fluorescent emission to the presence of the entity of interest in the assayed composition.

In some embodiments, the methods involve a quenching step in which the fluorescent nature of the kindled protein is quenched, e.g., by exposing the kindled protein to a quenching wavelength of light, as described above. Thus, in some embodiments, the methods comprise exposing the label to a kindling stimulus; exciting the kindled protein and detecting fluorescence emitted therefrom; and exposing the kindled protein to a quenching wavelength of light. For example, in particular embodiments, the methods include kindling a kindling fluorescent protein, e.g., with green light of a sufficient first intensity; exciting the kindled protein by exposure of the kindled KFP to green light of a lower intensity (e.g., 2-10,000 fold lower intensity); detecting any fluorescence from the kindled protein; and quenching the fluorescence with blue light. The kindling and quenching steps can be repeated any number of times. In particular embodiments, the methods include kindling a KFP with blue light; and detecting red fluorescence emitted by the kindled KFP upon exposure to green light.

The methods of the invention allow detection of very low levels of a kindling fluorescent protein. Thus, a kindling fluorescent protein can be detected when present in a sample at less than about 100, less than about 90, less than about 75, less than about 50, less than about 25, or less than about 10 molecules in a sample, down to a single molecule.

One specific application in which the subject protein/nucleic acid compositions find use is in protein labeling or tagging applications. In many of such applications, a protein of interest is labeled with a kindling protein by generating a fusion protein comprising the protein of interest and the kindling protein. Fusion proteins are produced using techniques well known to those skilled in the art.

Another specific application of interest is the labeling of a cell or a progeny of a cell by kindling a KFP produced in the cell (or a progeny of the cell), and detecting fluorescence of the kindled KFP and therefore the cell. In these embodiments, an expression construct that includes a nucleotide sequence that encodes the KFP is introduced into the cell, and after the KFP is produced in the cell, the KFP is kindled and detected.

Another specific application of interest includes use of the subject proteins/nucleic acid in methods of detecting the activity of a transcriptional and/or a translational regulatory element. A kindling fluorescent protein can thus be used to monitor the activity of a transcriptional and/or translational regulatory element. In these embodiments, a nucleic acid molecule comprising a nucleotide sequence that encodes a kindling fluorescent protein is operably linked to a regulatory element such that the kindling fluorescent protein is produced in a cell. Thus, a kindling fluorescent protein is used to monitor the activity of an enhancer, a promoter, or other regulatory elements. Similarly, a kindling fluorescent protein is useful to monitor the activity of a factor, such as a protein factor, that interacts with a promoter, enhancer, or other regulatory element. Applications using these methods are described in more detail below.

Representative applications are now described in greater detail. It should be noted that the following applications are provided as illustration only of the various and numerous different applications in which the subject invention finds use, and should therefore not be construed as limiting with respect to the scope of the claims attached hereto.

### Tracking Movement of KFPs

In some embodiments, movement of a KFP fused to a protein of interest is tracked. In other embodiments, movement of a KFP itself (e.g., a KFP not fused to a protein of interest) is tracked. In some embodiments, an expression vector that includes a nucleotide sequence encoding a KFP operably linked to a promoter is introduced into a cell. In other embodiments, mRNA encoding a KFP is introduced into a cell. In some of these embodiments, a nucleotide sequence encoding a KFP is operably linked to a promoter, such that the KFP is produced in a host cell, and the movement of the KFP is tracked. In some embodiments, the promoter is constitutive. In other embodiments, the promoter is conditionally active (e.g., inducible). The promoters can be regulated or constitutive, and in many instances are regulated (e.g., conditionally active). In some situations it may be desirable to use conditionally active promoters, including, but not limited to, tissue-specific promoters; cell type specific promoters; developmental stage-specific promoters; promoters controlled by the cell cycle; promoters controlled by Circadian rhythm; and promoters whose activity is increased (e.g., activated) or decreased (e.g., suppressed) in response to an external or internal signal. Many such promoters are well known in the art.

Movement of a KFP (e.g., tracking movement of a KFP protein or a KFP fused to a protein of interest; a subcellular structure labeled with a KFP; a cell labeled with a KFP) can be monitored in response to an external or internal signal, or in response to a test agent.

External and internal signals that affect protein movement include, but are not limited to, infection of a cell by a microorganism, including, but not limited to, a bacterium (e.g., Mycobacterium spp., Shigella, Chlamydia, and the like), a protozoan (e.g., Trypanosoma spp., Plasmodium spp., Toxoplasma spp., and the like), a fungus, a yeast (e.g., Candida spp.), or a virus (including viruses that infect mammalian cells, such as human immunodeficiency virus, foot and mouth disease virus, Epstein-Barr virus, and the like; viruses that infect plant cells; etc.); change in pH of the medium in which a cell is maintained or a change in internal pH; excessive heat relative to the normal range for the cell or the multicellular organism; excessive cold relative to the normal range for the cell or the multicellular organism; an effector molecule such as a hormone, a cytokine, a chemokine, a neurotransmitter; an ingested or applied drug; a ligand for a cell-surface receptor; a ligand for a receptor that exists internally in a cell, e.g., a nuclear receptor; hypoxia; light; dark; mitogens, including, but not limited to, lipopolysaccharide (LPS), pokeweed mitogen; antigens; sleep pattern (e.g., sleep deprivation, alteration in sleep pattern, and the like); electrical charge; ion concentration of the medium in which a cell is maintained or an internal ion concentration, exemplary ions including sodium ions, potassium ions, chloride ions, calcium ions, and the like; presence or absence of a nutrient; metal ions; a transcription factor; a tumor suppressor; cell-cell contact; and the like.

### Tracking Protein Movement

Kindling fluorescent proteins find use in applications for monitoring movement of a protein, and for monitoring protein turnover. Protein movement is monitored within a living cell (e.g., between organelles, between subcellular compartments, or within a cell membrane), i.e., intracellular movement; between two or more cells, i.e., intercellular movement; or from a living cell to an extracellular fluid (e.g., interstitial fluid, serum, cerebrospinal fluid, or other bodily fluid). Protein movement is monitored in vivo, within a living cell or group of cells, within a living tissue; during the course of development of a multicellular organism; in response to an external effect; in response to an ingested agent, and the like. Kindling fluorescent proteins are useful in investigations in which photobleaching techniques are currently employed. Thus, a kindling fluorescent protein can be used to label a protein to trace the movement of the protein within an intact cell, the nucleus, an organelle, or a cell membrane, from one cell to another, or from one location to another in a multicellular organism. Two prevalent photobleaching techniques are fluorescence recovery after photobleaching (FRAP) and fluorescence loss in photobleaching (FLIP). FRAP is used to estimate the turnover rates of molecules by the rate of influx of a bleached region by unbleached molecules from areas surrounding the bleached region. FRAP is described in numerous publications, including, e.g., Misteli et al. (2000) Nature 408:877-881; Snaar et al. (2000) J. Cell. Biol. 151:653-662; Dundr et al. (2000) J. Cell Biol 150:433-446; Estes et al. (2000) J. Neurogenet. 13 ;233-255 ; and Vos et al. (2000) Curr. Biol. 10:1-7. FLIP is based on monitoring the loss of fluorescence outside a repeatedly bleached region. FLIP studies show continuity of transport between different populations of fluorophores. FLIP has been described in the literature, including, e.g., in White and Stelzer (1999) Trends Cell Biol. 9:61-65, and references cited therein. The subject proteins can be used in analogous ways to the labeling agents employed in FRAP and FLIP assays, except for the fact that one is assaying for signal and not the absence of signal.

In some embodiments, the methods comprise exposing a defined or discrete portion of a cell containing a kindling fluorescent protein to a kindling wavelength of light. Defined portions of a cell include, but are not limited to, a mitochondria, a nucleus, a Golgi apparatus, an endoplasmic reticulum, a rough endoplasmic reticulum, a lysosome, a secretory vesicle, a chloroplast, and the like, or any part of these compartments. The kindled KFP (located in a portion of a cell irradiated by the KWL) is further traced using an excitation wavelength in the cell or within a group of cells, or within a tissue, by monitoring fluorescence. Studies of protein trafficking can be combined with studies of protein-protein interaction, using, e.g., FRET or BRET, which are described below. For example, trafficking of a first protein, protein X, can be monitored by creating a fusion protein between protein X (fusion protein X-KFP) and a kindling fluorescent protein. Protein X is known to interact with a second protein, protein Y. Protein Y is labeled with a GFP. In this scheme, the source of protein X may be organelle B or organelle A. Organelles A and B are independently exposed to a laser for a discrete period of time. Movement of fusion protein X-KFP, which now fluoresces, is monitored. When protein X encounters and binds to protein Y, the interaction of proteins X and Y can be detected using FRET.

Comparison of this FRET intensity when either A or B organelle were initially exposed to a laser shows the major source of the X, that interacts with Y (A or B). Other combinations of KFPs (with different kindling, excitation, emission wavelengths), FP (with different excitation, emission wavelengths), FRET (between different FP, KFP, dyes), photobleaching (of GFP-like proteins), etc. may be used in different experiments for protein trafficking.

In some applications, kindling fluorescent proteins are used to track the movement of a protein in a cell or an organism, e.g., a transgenic cell or organism that synthesizes a protein tagged with a kindling fluorescent protein, in response to exposure to a particular condition (e.g., an external or internal signal) or an agent. Conditions include, but are not limited to, various stresses, such as elevated temperature, hypoxia, and the like; periods of light and dark; sleep deprivation; and the like. Agents include, e.g., an agent being tested for therapeutic efficacy; an agent being tested for negative effects on a cell or an organism. The movement of the protein in the cell or organism exposed to a particular condition or agent is compared to a suitable control, e.g., a non-transgenic cell or organism of the same type; or a transgenic cell or organism not exposed to the test agent.

### Monitoring Movement of Subcellular Structures

KFPs are also useful to track movements of subcellular structures, e.g., an organelle. Organelles whose movement can be tracked using a KFP include, but are not limited to, mitochondria; Golgi apparatus; a membrane; a chloroplast; a vesicle; a vacuole; a lysosome; an endosome; endoplasmic reticulum (smooth or rough); and the like. In these embodiments, a KFP is expressed in the subcellular structure, and, at a given time, the organelle is exposed to a kindling stimulus. The subcellular structure's movement is tracked by exposing the organelle to an excitation wavelength. Movement of subcellular structure is monitored, e.g., in response to an external or internal signal, or in response to a test agent, as described above.

Non-limiting examples of a use of a KFP in tracking movement of subcellular structures include: (1) mitochondrial movement is in response to an apoptotic signal; (2) redistribution of subcellular structures during cell division; (3) vesicular transport; (4) axonal transport; (5) movement of endosomes in response to an antigen (e.g., processing of antigen through an endosome pathway); and the like.

### Monitoring Cellular Movement

KFPs are also useful to track movement of cells in a multicellular organism. In these embodiments, a KFP is expressed in a cell, and, at a given time, the cell is exposed to a kindling stimulus. The movement of the cell is monitored by exposing the cell to an excitation wavelength of light. The subject KFPs are useful for tracking cell migration in a multicellular organism, e.g., during embryonic development; during inflammation (e.g., to monitor lymphocyte homing; to monitor movement of macrophages); during the course of a normal immune response (e.g., to monitor lymphocyte homing); during the course of maturation of hematopoietic stem cells; during metastasis of a cancerous cell; and the like.

As one non-limiting example, the effectiveness of a treatment for cancer can be monitored in an experimental non-human animal by exposing cancer cells in vitro to a test agent, where the cancer cells produce a KFP and the KFP is kindled in vitro; introducing the cancer cells to the experimental animal; and monitoring the effect of the test agent on metastasis of the cancer cell by exposing the animal to an excitation wavelength. Alternatively, the KFP in the cancer cell is kindled after the cell is introduced into the animal.

As a further non-limiting example, the movement of a lymphocyte through a mammalian organism can be monitored by removing a lymphocyte from the organism, introducing an expression vector encoding a KFP into the lymphocyte in vitro, kindling the KFP, and reintroducing the lymphocyte into the same mammal. The movement of the lymphocyte is tracked by exposing the mammal to an excitation wavelength of light. Alternatively, the KFP in the cell can be kindled after the cell is reintroduced into the animal.

Tracking can be accomplished by kindling in vivo a KFP produced by a cell. Using fiber optics, a KFP produced by a cell is kindled in vivo. Using fiber optics, KFP produced by a discrete population of cells, or even a single cell, can be kindled, thereby allowing such cells to be monitored by detecting fluorescence. Fluorescence can be detected using a charge-coupled device camera. Thus, cell movement can be tracked without the need to dissect the animal. Use of CCD cameras to visualize reporter gene expression in whole animals is known in the art. See, e.g., Wu et al. (2002) Circulation 105:1631-1634.

A KFP that is a transgene expressed in a transgenic non-human animal or other organism (e.g., a transgenic plant) can be used to monitor movement of a cell in the organism. In some embodiments, the KFP is encoded by a nucleotide sequence that is operably linked to a regulatable (conditionally active) promoter. In some embodiments, the KFP is fused to a protein of interest. The transgenic non-human organism (or a part thereof is exposed to a kindling wavelength, and the movement of a cell producing the KFP is monitored. As one non-limiting example, the KFP transgene is operably linked to a regulatable promoter. The transgenic organism is exposed to a signal that activates the regulatable promoter, and the KFP is produced. The organism, or a part thereof, is exposed to a KWL and cell movement is monitored as described above. As another example, the KFP transgene is operably linked to a cell type-specific promoter such that the KFP is produced preferentially or exclusively in a particular cell type. Movement of the particular cell type is then monitored.

### Data Storage Applications

Kindling fluorescent proteins can be used as recording media for optical storage of information. Kindling fluorescent proteins find use in holography, digital and analog optical data storage, optical correlators, spatial light modulators, displays, optical switches, optical interconnects, and any other application in which the recording or control of light is required. See, for example, U.S. Pat. No. 20 6,046,925; U.S. Pat. No. 5,346,789; Irie, M. & Mori, M. J. Org. Chem. 53:803 (1988); Parthenopoulos, D. A. & Rentzepis, P. M. Science 245:843 (1989); Hanazawa, M., et al. J. Chem. Soc. Chem. Commun. 206 (1992); Dvornikov, A. S., et al. J. Phys. Chem. 98:6746-6752 (1994); Dvornikov, A. S. & Rentzepis, P. M. Opt. Mem. Neur. Netw. 3:75-86 (1994); U.S. Pat. No. 4,471,470; and U.S. Pat. No. 5,325,324. Kindling fluorescent proteins can be used in single-molecule optical storage, and multi-molecule optical storage. Kindling fluorescent proteins can be immobilized on a surface or on a matrix, such as PAA gel. Single-molecule optical storage can be achieved. See, W. E. Moerner, Science 265:46 (1994), in reference to low temperature devices. The device can function at room temperature because of the above-discussed temperature stability of kindling fluorescent proteins. Information can be stored by switching fluorescence of a kindling fluorescent protein “on” and “off” by irradiation with different wavelengths of light. Writing can be achieved by irradiating with a first, kindling wavelength of light. Erasing can be achieved with a second, quenching wavelength of light. Reading can be achieved by irradiating with low intensity excitation wavelength light, e.g. at an intensity below that at which kindling or quenching occurs. Similarly, writing and erasing can be achieved, for example, by irradiating with a second wavelength.

Different optical storage schemes can be implemented using a kindling fluorescent protein. First, two-dimensional, bit-oriented read/write/erase optical storage can be used. In this scheme, individual bits are addressed on a planar surface, i.e., by rotating a disk under a focusing lens which produces a diffraction-limited spot of light on the disk. Each bit is written by a different diffraction-limited spot on the disk. The diffraction-limited spot has a diameter on the order of the optical wavelength used for writing. With a mutant KFP, a recording layer can be made either by spin coating monomers on the surface of the disk before gelation or attaching the mutant KFP to a surface via a covalent bond (i.e., as a self-assembled monolayer). Alternatively, a recording layer can be fabricated by sandwiching a PAA gel containing the kindling fluorescent proteins between two transparent glass layers. Using a kindling fluorescent protein that has the property of kindling at a first wavelength of light and quenching at a second wavelength of light, the read/write operation can proceed using a first wavelength (e.g., green light) to write (and read), a second wavelength (e.g., blue light) to erase, or vice-versa. A second approach to optical storage is near-field optical storage. In this scheme, an optical device, like a pulled fiber tip or a solid immersion lens, is used to make a laser spot much smaller diffraction-limited spot (i.e., smaller than the optical wavelength). See, for example, Betzig, E. & Trautman, J. Science 257:189 (1992); and Terris, B. D., et al. Appl. Phys. Lett. 68:141 (1996). For example, a near-field fiber tip can generate an irradiation region that is about 50 nm in diameter. The solid immersion lens is more robust, but it produces a spot approximately 300 nm in diameter. In either case, much higher areal densities can be achieved than with diffraction-limited spots. For this type of storage, the recording layer must be very close to either the tip or the immersion lens. The kindling fluorescent protein can be reactively attached to a fused silica or glass surface that has been silanized and made functional for attachment to free amines of, for example, lysine, or to sulfhydryl functionality of cysteine amino acids. By patterning of the surface functionalization, single kindling fluorescent protein molecules can be distributed in an array on the surface.

A third approach to optical storage is holographic optical storage. In this scheme, higher storage densities can be achieved by using an optically thick hologram for the recording. The actual recording is made by locally changing the refractive index or the optical absorption in the material. Read/write volume holographic storage based on photorefractive materials is generally described in Sincerbox, G. T. Optical Materials 4:370 (1995). A recent demonstration using LiNbO₃ is described in Heanue, J. F. et al. Science 265:749 (1994). A review of holographic memory using general materials, including (write-once) photopolymers is given in D. Psaltis and F. Mok, Scientific American November 1995,. p. 70. In holographic storage, an image-bearing beam and a reference beam are both impressed upon the material, and the change in the optical properties ensues at the locations in the material bearing high intensities of irradiation to record an image. After recording, when only the reference beam is turned on, a diffracted beam appears which is a copy of the original image beam. To erase the stored image, a uniform beam is left on for a longer period of time. To achieve high information density, many holograms can be written in the same volume by using a different angle, phase, or frequency of reference beam for each image. Holographic optical storage can provide high data transfer rates by parallel readout, and high density of storage.

Kindling fluorescent proteins can be embedded in a thick gel material, and two laser beams at a first wavelength can be used to write a hologram in the gel. The hologram can be read with illumination at the excitation wavelength by fluorescence or by diffraction. The hologram can be erased by irradiation at a second wavelength. Alternatively, the material can be irradiated uniformly with a first wavelength to drive all the molecules into the N state. Subsequent irradiation with two beams at a second wavelength can produce a hologram in the gel. Erasure in this case can require exposure to a different wavelength.

A fourth approach to optical storage is two-photon volumetric optical storage. Photochromic materials for two-photon three dimensional optical storage have been reviewed by Dvornikov, A. S., et al. J. Phys. Chem. 98:6746 (1994), and by Dvornikov, A. S. & Rentzepis, P. M. Opt. Mem. Neur. Netw. 3:75 (1994). See also, for example, U.S. Pat. No. 5,325,324. In this scheme, localization of a written bit in a three dimensional medium can be achieved by using a nonlinear interaction with the material, such as a two-photon optical excitation. A laser beam of long wavelength and high intensity can be used to pump a two-photon transition, which causes a change in the optical properties of the material. The intensity dependence is I-squared or higher. The two-photon approach results in a high degree of localization of the altered region in all three dimensions. Next, a second two-photon process can be used for readout (e.g., by fluorescence). Erasing of the medium can be done by one-photon irradiation of the medium at another wavelength. One advantage of a two-photon mechanism is that essentially erasing does not occur during reading.

### Other Applications

Kindling fluorescent proteins find use in a variety of additional applications. One application of interest is the use of the kindling fluorescent proteins as detectable labels which are capable of imparting fluorescence to a particular composition of matter. Of particular interest in certain embodiments are non-toxic kindling fluorescent proteins. Kindling fluorescent proteins may be incorporated into a variety of different compositions of matter, where representative compositions of matter include: food compositions, pharmaceuticals, cosmetics, living organisms, e.g., animals and plants, and the like. Where used as a detectable label, a sufficient amount of the kindling fluorescent protein is incorporated into the composition of matter to impart the desired fluorescence thereto. The kindling fluorescent protein may be incorporated into the composition of matter using any convenient protocol, where the particular protocol employed will necessarily depend, at least in part, on the nature of the composition of matter to be labeled. Protocols that may be employed include, but are not limited to: blending, diffusion, friction, spraying, injection, tattooing, and the like.

The kindling fluorescent proteins may also find use as labels in analyte detection assays, e.g., assays for biological analytes of interest. For example, the kindling fluorescent proteins may be incorporated into adducts with analyte specific antibodies or binding fragments thereof and subsequently employed in immunoassays for analytes of interest in a complex sample, as described in U.S. Pat. No. 4,302,536; the disclosure of which is herein incorporated by reference. Instead of antibodies or binding fragments thereof, kindling fluorescent proteins or fluorescent fragments thereof may be conjugated to ligands that specifically bind to an analyte of interest, or other moieties, growth factors, hormones, and the like; as is readily apparent to those of skill in the art.

In yet other embodiments, the subject kindling fluorescent proteins may be used as selectable markers in recombinant DNA applications, e.g., the production of transgenic cells and organisms, as described above. As such, one can engineer a particular transgenic production protocol to employ expression of the subject kindling fluorescent proteins as a selectable marker, either for a successful or unsuccessful protocol. Thus, appearance of the color of the subject kindling fluorescent proteins in the phenotype of the transgenic organism produced by a particular process can be used to indicate that the particular organism successfully harbors the transgene of interest, often integrated in a manner that provides for expression of the transgene in the organism. When used a selectable marker, a nucleic acid encoding a kindling fluorescent protein can be employed in the transgenic generation process, where this process is described in greater detail supra. Particular transgenic organisms of interest where a kindling fluorescent protein may be employed as a selectable marker include transgenic plants, animals, bacteria, fungi, and the like.

Kindling fluorescent proteins find use in fluorescence resonance energy transfer (FRET) applications. In these applications, kindling fluorescent proteins serve as donor and/or acceptors in combination with a second fluorescent protein or dye, e.g., a fluorescent protein as described in Matz et al., Nature Biotechnology (October 1999) 17:969-973, a green fluorescent protein from Aequoria victoria or fluorescent mutant thereof, e.g., as described in U.S. Pat. No. 6,066,476; 6,020,192; 5,985,577; 5,976,796; 5,968,750; 5,968,738; 5,958,713; 5,919,445; 5,874,304, the disclosures of which are herein incorporated by reference, other fluorescent dyes, e.g., coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green,. rhodamine dyes, e.g. texas red, tetramethylrhodamine, eosins and erythrosins, cyanine dyes, e.g. Cy3 and Cy5, macrocyclic chelates of lanthanide ions, e.g. quantum dye, etc., chemilumescent dyes,.e.g., luciferases, including those described in U.S. Pat. Nos. 5,843,746; 5,700,673; 5,674,713; 5,618,722; 5,418,155; 5,330,906; 5,229,285; 5,221,623; 5,182,202; the disclosures of which are herein incorporated by reference.

Specific examples of where FRET assays employing kindling fluorescent proteins may be used include, but are not limited to: the detection of protein-protein interactions, e.g., mammalian two-hybrid system, transcription factor dimerization, membrane protein multimerization, multiprotein complex formation, etc., as a biosensor for a number of different events, where a peptide or protein covalently links a FRET fluorescent combination including the subject fluorescent proteins and the linking peptide or protein is, e.g., a protease specific substrate, e.g., for caspase mediated cleavage, a linker that undergoes conformational change upon receiving a signal which increases or decreases FRET, e.g., PKA regulatory domain (cAMP-sensor), phosphorylation, e.g., where there is a phosphorylation site in the linker or the linker has binding specificity to phosphorylated/dephosphorylated domain of another protein, or the linker has Ca²⁺ binding domain. Representative fluorescence resonance energy transfer or FRET applications in which the subject proteins find use include, but are not limited to, those described in: U.S. Pat. Nos. 6,008,373; 5,998,146; 5,981,200; 5,945,526; 5,945,283; 5,911,952; 5,869,255; 5,866,336; 5,863,727; 5,728,528; 5,707,804; 5,688,648; 5,439,797; the disclosures of which are herein incorporated by reference.

Another application in which kindling fluorescent proteins find use is BRET (Bioluminescence Resonance Energy Transfer). BRET is a protein-protein interaction assay based on energy transfer from a bioluminescent donor to a fluorescent acceptor protein. The BRET signal is measured by the amount of light emitted by the acceptor to the amount of light emitted by the donor. The ratio of these two values increases as the two proteins are brought into proximity. The BRET assay has been amply described in the literature. See, e.g., U.S. Pat. Nos. 6,020,192; 5,968,750; and 5,874,304; and Xu et al. (1999) Proc. Natl. Acad. Sci. USA 96:151-156. BRET assays may be performed by genetically fusing a bioluminescent donor protein and a fluorescent acceptor protein independently to two different biological partners to make partner A-bioluminescent donor and partner B-fluorescent acceptor fusions. Changes in the interaction between the partner portions of the fusion proteins, modulated, e.g., by ligands or test compounds, can be monitored by a change in the ratio of light emitted by the bioluminescent and fluorescent portions of the fusion proteins. In this application, the subject proteins serve as donor and/or acceptor proteins. BRET assays can be used in many of the assays as FRET, which assays are noted above.

Kindling fluorescent proteins also find use as biosensors in prokaryotic and eukaryotic cells, e.g. as Ca²⁺ ion indicator; as pH indicator, as phorphorylation indicator, as an indicator of other ions, e.g., magnesium, sodium, potassium, chloride and halides. For example, for detection of Ca ion, proteins containing an EF-hand motif are known to translocate from the cytosol to membranes upon Ca²+ binding. These proteins contain a myristoyl group that is burried within the molecule by hydrophobic interactions with other regions of the protein. Binding of Ca²+ induces a conformational change exposing the myristoyl group which then is available for the insertion into the lipid bilayer (called a “Ca²+ -myristoyl switch” ). Fusion of such an EF-hand containing protein to Fluorescent Proteins (FP) could make it an indicator of intracellular Ca²⁺ by monitoring the translocation from the cytosol to the plasma membrane by confocal microscopy. EF-hand proteins suitable for use in this system include, but are not limited to: recoverin (1-3), calcineurin B, troponin C, visinin, neurocalcin, calmodulin, parvalbumin, and the like. For pH, a system based on hisactophilins may be employed. Hisactophilins are myristoylated histidine-rich proteins known to exist in Dictyostelium. Their binding to actin and acidic lipids is sharply pH-dependent within the range of cytoplasmic pH variations. In living cells membrane binding seems to override the interaction of hisactophilins with actin filaments. At pH<6.5 they locate to the plasma membrane and nucleus. In contrast, at pH 7.5 they evenly distribute throughout the cytoplasmic space. This change of distribution is reversible and is attributed to histidine clusters exposed in loops on the surface of the molecule. The reversion of intracellular distribution in the range of cytoplasmic pH variations is in accord with a pK of 6.5 of histidine residues. The cellular distribution is independent of myristoylation of the protein.

By fusing FPs (Fluoresent Proteins) to hisactophilin the intracellular distribution of the fusion protein can be followed by laser scanning, confocal microscopy or standard fluorescence microscopy. Quantitative fluorescence analysis can be done by performing line scans through cells (laser scanning confocal microscopy) or other electronic data analysis (e.g., using metamorph software (Universal Imaging Corp) and averaging of data collected in a population of cells. Substantial pH-dependent redistribution of hisactophilin-FP from the cytosol to the plasma membrane occurs within 1-2 min and reaches a steady state level after 5-10 min. The reverse reaction takes place on a similar time scale. As such, hisactophilin-fluorescent protein fusion protein that acts in an analogous fashion can be used to monitor cytosolic pH changes in real time in live mammalian cells. Such methods have use in high throughput applications, e.g., in the measurement of pH changes as consequence of growth factor receptor activation (e.g. epithelial or platelet-derived growth factor) chemotactic stimulation/cell locomotion, in the detection of intracellular pH changes as second messenger, in the monitoring of intracellular pH in pH manipulating experiments, and the like. For detection of PKC activity, the reporter system exploits the fact that a molecule called MARCKS (myristoylated alanine-rich C kinase substrate) is a PKC substrate. It is anchored to the plasma membrane via myristoylation and a stretch of positively charged amino acids (ED-domain) that bind to the negatively charged plasma membrane via electrostatic interactions. Upon PKC activation the ED-domain becomes phosphorylated by PKC, thereby becoming negatively charged, and as a consequence of electrostatic repulsion MARCKS translocates from the plasma membrane to the cytoplasm (called the “myristoyl-electrostatic switch” ). Fusion of the N-terminus of MARCKS ranging from the myristoylation motif to the ED-domain of MARCKS to fluorescent proteins of the present invention makes the above a detector system for PKC activity. When phosphorylated by PKC, the fusion protein translocates from the plasma membrane to the cytosol. This translocation is followed by standard fluorescence microscopy or confocal microscopy e.g. using the Cellomics technology or other High Content Screening systems (e.g. Universal Imaging Corp./Becton Dickinson).

The above reporter system has application in High Content Screening, e.g., screening for PKC inhibitors, and as an indicator for PKC activity in many screening scenarios for potential reagents interfering with this signal transduction pathway. Methods of using fluorescent proteins as biosensors also include those described in U.S. Pat. Nos. 972,638; 5,824,485 and 5,650,135 (as well as the references cited therein) the disclosures of which are herein incorporated by reference.

Kindling fluorescent proteins also find use in applications involving the automated screening of arrays of cells expressing fluorescent reporting groups by using microscopic imaging and electronic analysis. Screening can be used for drug discovery and in the field of functional genomics: e.g., where the subject proteins are used as markers of whole cells to detect changes in multicellular reorganization and migration, e.g., formation of multicellular tubules (blood vessel formation) by endothelial cells, migration of cells through Fluoroblok Insert System (Becton Dickinson Co.), wound healing, neurite outgrowth, etc.; where the proteins are used as markers fused to peptides (e.g., targeting sequences) and proteins that allow the detection of change of intracellular location as indicator for cellular activity, for example: signal transduction, such as kinase and transcription factor translocation upon stimuli, such as protein kinase C, protein kinase A, transcription factor NFkB, and NFAT; cell cycle proteins, such as cyclin A, cyclin B1 and cyclinE; protease cleavage with subsequent movement of cleaved substrate, phospholipids, with markers for intracellular structures such as endoplasmic reticulum, Golgi apparatus, mitochondria, peroxisomes, nucleus, nucleoli, plasma membrane, histones, endosomes, lysosomes, microtubules, actin) as tools for High Content Screening: co-localization of other fluorescent fusion proteins with these localization markers as indicators of movements of intracellular fluorescent fusion proteins/peptides or as marker alone; and the like. Examples of applications involving the automated screening of arrays of cells in which the subject fluorescent proteins find use include: U.S. Pat. No. 5,989,835; as well as WO/0017624; WO 00/26408; WO 00/17643; and WO 00/03246; the disclosures of which are herein incorporated by reference.

Kindling fluorescent proteins also find use in high through-put screening assays. Kindling fluorescent proteins are stable proteins with half-lives of more than 24 h. Also provided are destabilized versions of the subject fluorescent proteins with shorter half-lives that can be used as transcription reporters for drug discovery. For example, a kindling fluorescent protein can be fused with a putative proteolytic signal sequence derived from a protein with shorter half-life, e.g., PEST sequence from the mouse ornithine decarboxylase gene, mouse cyclin B1 destruction box and ubiquitin, etc. Promoters in signal transduction pathways can be detected using destabilized versions of the subject fluorescent proteins for drug screening, e.g., AP1, NFAT, NFkB, Smad, STAT, p53, E2F, Rb, myc, CRE, ER, GR and TRE, and the like.

Kindling fluorescent proteins can be used as second messenger detectors, e.g., by fusing the subject proteins to specific domains: e.g., PKCgamma Ca binding domain, PKCgamma DAG binding domain, SH2 domain and SH3 domain, etc.

Secreted forms of kindling fluorescent proteins can be prepared, e.g. by fusing secreted leading sequences to the subject proteins to construct secreted forms of kindling fluorescent proteins, which in turn can be used in a variety of different applications.

Kindling fluorescent proteins also find use in fluorescence activated cell sorting applications. In such applications, a kindling fluorescent protein is used as a label to mark a population of cells and the resulting labeled population of cells is then sorted with a fluorescent activated cell sorting device, as is known in the art. FACS methods are described in U.S. Pat. Nos. 5,968,738 and 5,804,387; the disclosures of which are herein incorporated by reference.

Kindling fluorescent proteins also find use as in vivo marker in animals (e.g., transgenic animals). For example, expression of a kindling fluorescent protein can be driven by tissue specific promoters, where such methods find use in research for gene therapy, e.g., testing efficiency of transgenic expression, among other applications. A representative application of kindling fluorescent proteins in transgenic animals that illustrates this class of applications of the subject proteins is found in WO 00/02997, the disclosure of which is herein incorporated by reference.

Additional applications of kindling fluorescent proteins include: as markers following injection into cells or animals and in calibration for quantitative measurements (fluorescence and protein); as markers or reporters in oxygen biosensor devices for monitoring cell viability; as markers or labels for animals, pets, toys, food, etc.; and the like.

Kindling fluorescent proteins also find use in protease cleavage assays. For example, cleavage inactivated fluorescence assays can be developed using kindling fluorescent proteins, where kindling fluorescent proteins are engineered to include a protease specific cleavage sequence without destroying the fluorescent character of the protein. Upon cleavage of the kindling fluorescent protein by an activated protease fluorescence would sharply decrease due to the destruction of a functional chromophore. Alternatively, cleavage activated fluorescence can be developed using the subject proteins, where the kindling fluorescent proteins are engineered to contain an additional spacer sequence in close proximity/or inside the chromophore. This variant would be significantly decreased in its fluorescent activity, because parts of the functional chromophor would be divided by the spacer. The spacer would be framed by two identical protease specific cleavage sites. Upon cleavage via the activated protease the spacer would be cut out and the two residual “subunits” of the fluorescent protein would be able to reassemble to generate a functional fluorescent protein. Both of the above types of application could be developed in assays for a variety of different types of proteases, e.g., caspases, etc.

Kindling fluorescent proteins can also be used is assays to determine the phospholipid composition in biological membranes. For example, fusion proteins of kindling fluorescent proteins (or any other kind of covalent or non-covalent modification of kindling fluorescent proteins) that allows binding to specific phospholipids to localize/visualize patterns of phospholipid distribution in biological membranes also allowing colocalization of membrane proteins in specific phospholipid rafts can be accomplished with kindling fluorescent proteins. For example, the PH domain of GRP1 has a high affinity to phosphatidyl-inositol tri-phosphate (PIP3) but not to PIP2. As such, a fusion protein between the PH domain of GRP1 and a kindling fluorescent protein can be constructed to specifically label PIP3 rich areas in biological membranes.

The antibodies of the subject invention, described above, also find use in a number of applications, including the differentiation of kindling fluorescent proteins from other fluorescent proteins.

### Systems

Also provided are systems for using in practicing the subject methods. The subject systems typically include: (a) a kindling fluorescent protein composition or nucleic acid composition encoding the same, e.g., a vector including a nucleic acid encoding a kindling fluorescent protein; (b) and a kindling stimulus source, such as the sources described above. The systems can also include an excitation source and fluorescence detector, etc. In addition, the subject systems also include any reagents employed in a given assay in which the subject systems are being used.

### Kits

Also provided by the subject invention are kits for use in practicing one or more of the above described applications, where the subject kits typically include elements for making the subject proteins, e.g., a construct comprising a vector that includes a coding region for the subject protein. The subject kit components are typically present in a suitable storage medium, e.g., buffered solution, typically in a suitable container. Also present in the subject kits may be antibodies to the provided protein. In certain embodiments, the kit comprises a plurality of different vectors each encoding the subject protein, where the vectors are designed for expression in different environments and/or under different conditions, e.g., constitutive expression where the vector includes a strong promoter for expression in mammalian cells, a promoterless vector with a multiple cloning site for custom insertion of a promoter and tailored expression, etc.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustrations and not by way of limitation.

### Experimental

I. Generation of and Initial Characterization of Kindling Fluorescent Proteins

A. Materials & Methods

1. Parent Proteins

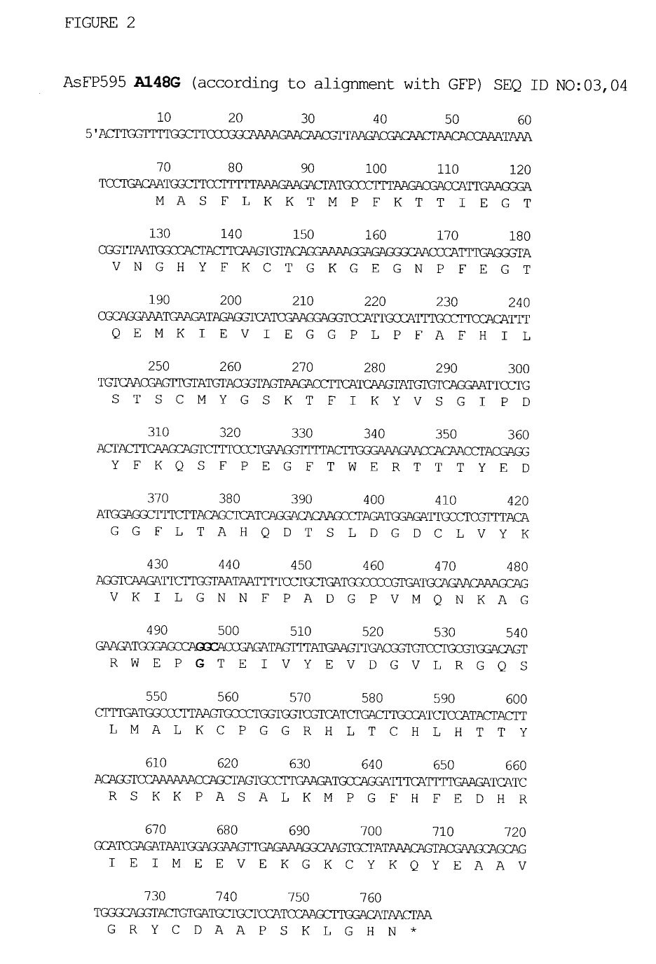

Routine target, i.e., site specific, and random mutagenesis of wild type asFP595 and H. crispa chromo proteins was carried out. The asFP595 (asCP) amino acid sequence and a nucleotide coding sequence therefore are provided in FIG. 1 (i.e., SEQ ID NOs: 01 and 02). This protein is further described in U.S. patent application Ser. No.10/006,922, the disclosure of which is herein incorporated by reference. The H.crispa chromoprotein amino acid sequence and a nucleotide coding sequence therefore are provided in FIG. 2 (i.e., SEQ ID NOs: 07 and 08). This protein is further described in U.S. patent application Ser. No. 09/976,673, the disclosure of which is herein incorporated by reference.

2. Target Mutagenesis

Mutagenesis was performed by the overlap extension method (Ho et al. (1989) Gene 77, 51 (1989). Briefly, two overlapping fragments of each FP coding region were amplified. PCR was carried out using Advantage® 2 Polymerase Mix (CLONTECH) in 1×manufacturer's buffer supplemented with 100 μM of each dNTP, 0.2 μM of each primer and 1 ng of plasmid DNA in 25 μl (final volume). To remove plasmids encoding wild type proteins, the 5′- and 3′-fragments were excised from 2% low-melting agarose gel in 1×TAE buffer. To drain the DNA solution, the gel pieces were subjected to 3 freeze-thaw cycles. For each particular mutant, appropriate 5′- and 3′-fragments were combined to obtain full-length cDNA as follows. Equal volumes of 5′-fragment solution, 3′-fragment solution and 3×PCR mixture containing Advantage 2 Polymerase Mix, buffer and dNTPs were mixed together and subjected to 2-3 cycles of 95° C. for 20 s, 65° C. for 30 min, 72° C. for 30 s. Then, the reaction was diluted 10 fold and 1 μl of the diluted sample was used as a template for PCR with forward and reverse cloning primers (as described above for 5′- and 3′-fragments amplification). As a result, ready-for-cloning fragments containing full-length coding regions with target substitution(s) were generated.

3. Cloning

Mutant PCR products were digested with endonucleases, for which the cloning primers contain sites, and then cloned into pQE30 (Qiagen) digested with endonucleases generating complementary overhangs. Each of the recombinant proteins generated by both cloning-expression systems contained a 6×His tag on the N- or C-terminus.

4. Protein Purification

Selected E. coli clones were grown at 37° C. in 50 ml to an optical density of (OD) 0.6. At that point, the expression of recombinant FP was induced with 0.2 mM IPTG. The cultures were then incubated overnight. The following day, cells were harvested by centrifugation, resuspended in buffer (20 mM Tris-HCl, pH 8.0; 100 mM NaCl), and disrupted by sonication. Fluorescent proteins were purified from the soluble fraction using TALON Metal Affinity Resin (CLONTECH). Proteins were at least 95% pure according to SDS-PAGE.

5. Spectroscopy.

The concentrations of the purified proteins were determined by measuring absorbance at 280 nm and the extinction coefficients were calculated as described in [Mach H, et al., Statistical determination of the average values of the extinction coefficients of tryptophan and tyrosine in native proteins. Anal Biochem. 1992 Jan; 200(1):74-80]. These data and the absorption spectra were used to calculate molar extinction coefficients for absorption bands in the visible portion of the spectra. Quantum yields were determined relative to wild type GFP (CLONTECH Laboratories, Inc., Palo Alto, Calif.). A Perkin-Elmer LS50B spectrometer was used for quantitative measurements. The spectra were corrected for photomultiplier response and monochromator transmittance, transformed to wave number and integrated. To compare the different proteins and their mutants, the intrinsic fluorescence brightness of each protein was calculated as the maximum extinction coefficient multiplied by the quantum yield.

B. Results

A series of mutants based on AsFP595 (FP7) and H. crispa (FP10) were generated and characterized using the methods described above. The properties of these mutants are summarized in Table 1. “Fast” indicates that kindling or quenching occurred over a period of up to about 60 seconds. “Very fast” indicates that kindling or quenching occurred over a period of less than about 15 seconds. The corresponding sequence identification numbers are also provided, where “nt” indicates “polynucleotide sequence” and “aa” indicates “amino acid sequence” sequence identifiers.

TABLE 1

| | Parent | | Kindling; | Quenching; | Excit/Emiss | Relaxation | SEQ ID |
|---|---|---|---|---|---|---|---|
| Protein | protein | Mutation(s)* | Speed | Speed | Peaks, nm | time, min. | NOs nt;aa |
| KFP 04a | AsFP595 | A148G | Green; | Blue; very | 570/ | appr. 15 | 03;04 |
| (KFP 1) | | | fast | fast | 595 | | |
| KFP 04b | AsFP595 | F90L; A148G; | Green; | Blue; very | 570/ | appr. 30 | 05;06 |
| | | H203Y | fast | fast | 595 | | |
| KFP 08a | H. crispa | K28M; N165A | Blue; very | No | 590/ | appr. 1.5 | 09;10 |
| | | | fast | | 630 | | |
| KFP 08b | H. crispa | K28M; N165G | Blue; very | No | 590/ | appr. 0.3 | 11;12 |
| | | | fast | | 620 | | |
| KFP 08c | H. crispa | G20C,T39A; | Green; | No | 590/ | appr. 30 | 13;14 |
| | | L126H | fast | | 620 | | |
| | | C148A; | | | | | |
| | | N165G; | | | | | |
| | | R176H; | | | | | |
| | | L181H; | | | | | |
| | | A190V; | | | | | |
| | | P208L; | | | | | |
| | | K211E | | | | | |
| KFP 08d | H. crispa | T39A; C148S; | Green; | No | 590/ | appr. 30 | 15;16 |
| | | N165S; | fast | | 620 | | |
| | | L181H; | | | | | |
| | | I203H; | | | | | |
| | | P208R; | | | | | |
| | | K211E | | | | | |

*Mutation positions determined using the GFP alignment protocol as described in Matz et al., Nature Biotech. (1999)) 17:969-973.

Most of the mutant KFPs were capable of irreversible kindling, if irradiated very intensively (i.e., HeNe laser line (543 nm, 1 mW), 30% power, Carl Zeiss Confocal Laser Scanning Microscope LSM 510, 100×objective) for short period of time (from 0.001 second to several sec) or intensively (using a TRITC filter set on a Polyvar microscope (Reihert-Jung) through a ×40 objective, 100 watt lamp) for several minutes. In some embodiments, irreversible kindling can still be fully or partially quenched by an appropriate quenching light, even several days after the kindling event.

Each of the above proteins and mutants is more fully characterized as follows:

1. Wild Type asFP595

asFP595 has the following properties: (1) Non-kindled state: nonfluorescent; kindling stimulus: green light of medium intensity, e.g., at least about 0.1-1 W/cm², (low intensity green light causes no kindling); (2) Kindled state: brightly fluorescent but not kindles in green light of low intensity, e.g., intensity less than about 0.1-0.01 W/cm²; (3) This KFP, having been kindled, relaxes back to the nonfluorescent state after about 1 min visible); (4) Kindled fluorescence is quenched upon exposure to blue light; (5) Quenching is reversible. Thus, it is possible to turn the fluorescence of asFP595 on and off multiple times by exposure to different wavelengths of light. Irreversible kindling is detected for asFP595 in ultraviolet light (about 250-350 nm).

2. Mutants of Wild Type asFP595

a. KFP-04a (KFP1)(asFP595-mutant 1) (asFP595, A148G, according to alignment with GFP; amino acid sequence as set forth in SEQ ID NO:04).

AsFP595-mutant 1 (also referred to as KFP-04a) has the following properties: (1) Non-kindled state: nonfluorescent or weakly fluorescent; (2) Kindling stimulus: green light of medium intensity of at least about 0.1-1 W/cm² (low intensity green light causes no kindling); (3) Kindled state: brightly fluorescent but not kindles in green light of low intensity of less than about 0.1-0.01 W/cm²; (4) Contrast between kindled and unkindled KFP-04a fluorescence intensity is more than 50 times; (5) This KFP, having been kindled, relaxes back to the nonfluorescent state after about 15 minutes visible; (6) Kindled fluorescence is quenched upon exposure to blue light; (7) Quenching is reversible. Thus, it is possible to turn the fluorescence of a KFP-04a on and off multiple times by exposure to different wavelengths of light. Very intensive/durative green light irradiation of at least about 1-10 W/cm² causes irreversible kindling of KFP-04a. The contrast between irreversibly kindled and unkindled KFP-04a fluorescence intensity is more than 30 times. After irreversible kindling, partial and temporary quenching is still possible, even after several weeks, or months, after the kindling event. Irreversible kindling is detected for KFP-04a in ultraviolet light (about 250-350 nm).

b. KFP-04b (asFP595-mutant 2) (asFP595, F90L, A148G, H203Y, according to alignment with GFP; amino acid sequence as set forth in SEQ ID NO:06).

asFP595-mutant 2 (also referred to as KFP-04b) has the following properties: (1) Non-kindled state: nonfluorescent; (2) kindling stimulus: green light of medium intensity of at least about 0.1-1 W/cm² (low intensity green light having an intensity of less than about 0.1-0.01 W/cm² causes no kindling); (3) Kindled state: brightly fluorescent in green light of low intensity of less than about 0.1-0.01 W/cm²; (4) This KFP, having been kindled, relaxes back to the nonfluorescent state after about 30 minutes visible); (5) No fluorescence quenching for this KFP is detected; (6) Very intensive/durative green light irradiation of at least about 1 W/cm² causes irreversible kindling of KFP-04b.

3. Mutants of H. crispa chromoprotein, wild type; SEQ ID NO:08.

Specific mutants of interest include the following.

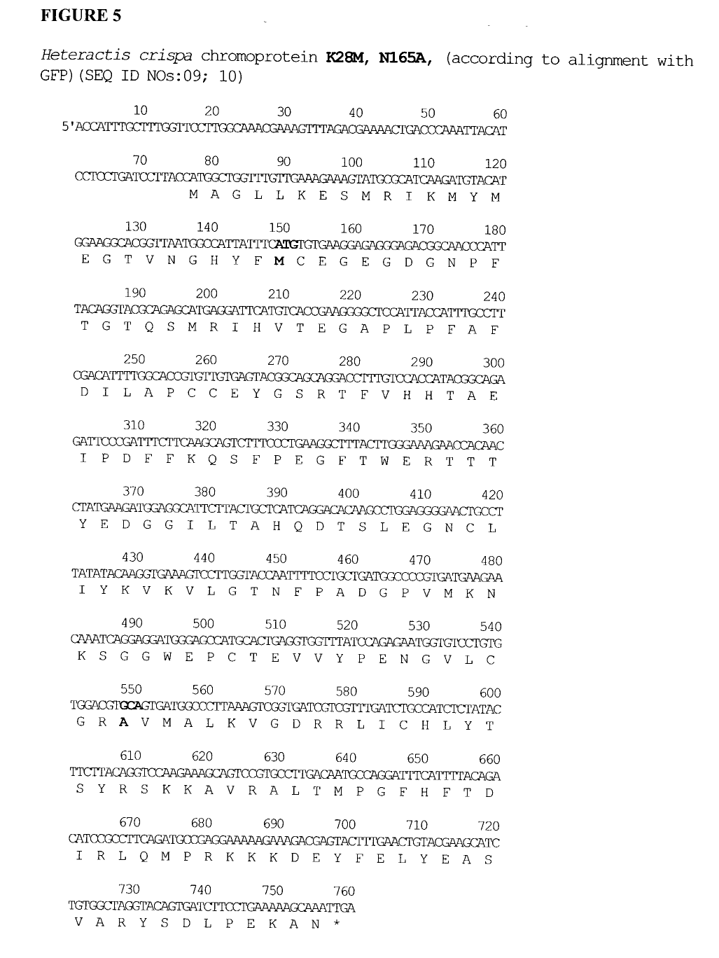

a. KFP-08a (H. crispa mutant 1) (H. crispa chromoprotein, K28M, N165A, according to alignment with GFP; amino acid sequence depicted in FIG. 5 and set forth in SEQ ID NO:10).

H. crispa mutant 1 (also referred to as KFP-08a) has the following properties: (1) Non-kindled state: nonfluorescent or weakly fluorescent; (2) kindling stimulus: blue light of medium or low intensity, e.g., that ranges from about 0.01 W/cm² to about 10 W/cm²; (3) Kindled state: brighly fluorescent in green light; (4) This KFP, having been kindled, relaxes back to the nonfluorescent state after about 1 minute; (5) Very intensive/durative blue light irradiation of at least about 1W/cm² causes irreversible kindling of KFP-08a. Irreversible kindling is detected for KFP -08a in ultraviolet light (about 250-350 nm).

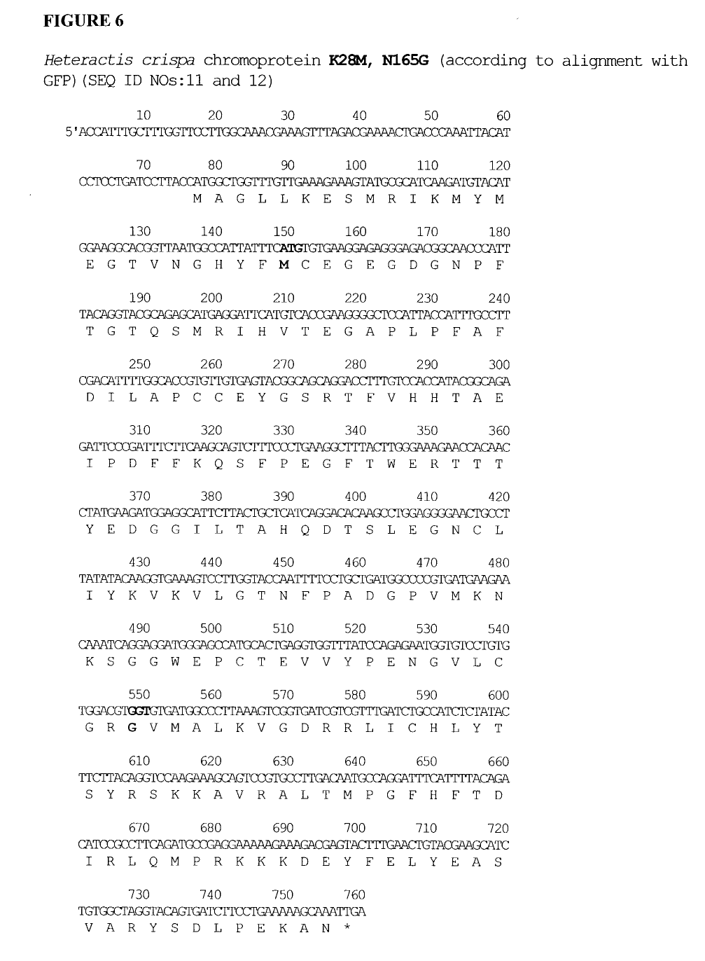

b. KFP-08b (H. crispa mutant 2;H. crispa chromoprotein, K28M, N165G, according to alignment with GFP; amino acid sequence depicted in FIG. 6 and set forth in SEQ ID NO:12).

H. crispa mutant 2 (also referred to as KFP08b) has the following properties: (1) Non-kindled state: nonfluorescent or weakly fluorescent; (2) kindling stimulus: blue light of medium or low intensity of from about 0.01 W/cm² to about 10 W/cm²; (3) Kindled state: brightly fluorescent in green light; (4) This KFP, having been kindled, relaxes back to the nonfluorescent state after about 10 seconds; (5) Very intensive/durative blue light irradiation of at least about 1W/cm² causes irreversible kindling of KFP-08b.

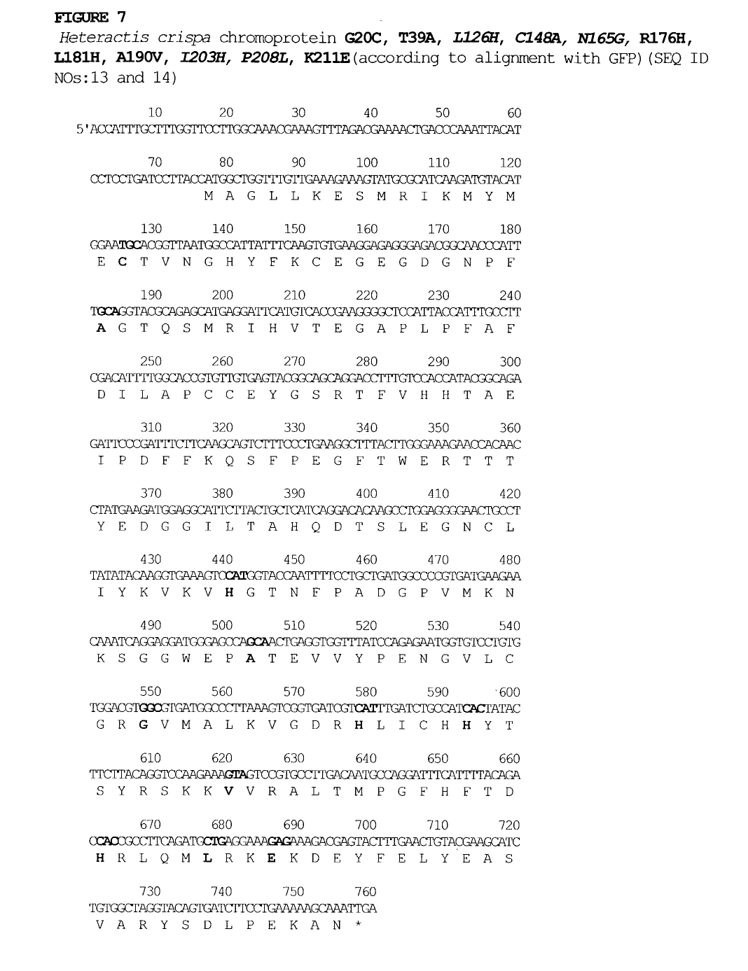

c. KFP-08c (H. crispa mutant 3; H. crispa chromoprotein, G20C, T39A, L126H, C148A, N165G, R176H, L181H, A190V, I203H, P208L, K211E, according to alignment with GFP; amino acid sequence depicted in FIG. 7 and set forth in SEQ ID NO:14).

H. crispa mutant 3 (also referred to as KFP-08c) has the following properties: (1) Non-kindled state: nonfluorescent or weakly fluorescent; (2) kindling stimulus: green light of medium intensity of at least about 0.1-1 W/cm²; (3) Kindled state: brighly fluorescent in green light of low intensity of less than about 0.1-0.01 W/cm²; (4) This KFP, having been kindled, relaxes back to the nonfluorescent state after about 30 minutes; (5) No fluorescence quenching for this KFP is detected; (6) KFP-08c exists primarily as a dimer; (6) Very intensive/durative green light irradiation causes irreversible kindling of KFP-08c.

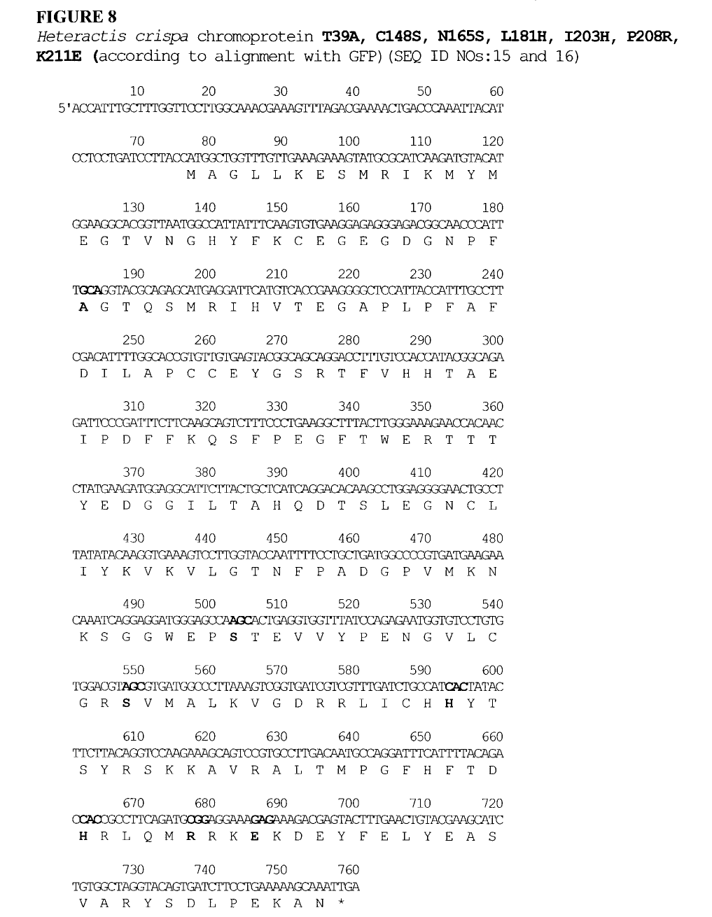

d. KFP-08d (H. crispa mutant 4;H. crispa chromoprotein, T39A, C148S, N165S, L181H, I203H, P208R, K211E according to alignment with GFP; amino acid sequence depicted in FIG. 8 and set forth in SEQ ID NO:16).

H. crispa mutant 4 (also referred to as KFP-08d) has the following properties: (1) Non-kindled state: nonfluorescent or weakly fluorescent; (2) kindling stimulus: green light of medium intensity of at least about 0.1-1 W/cm²; (3) Kindled state: brighly fluorescent in green light of low intensity of less than about 0.1-0.01 W/cm²; (4) This KFP, having been kindled, relaxes back to the nonfluorescent state after about 30 minutes; (5) No fluorescence quenching for this KFP is detected; (6) Very intensive/durative green light irradiation causes irreversible kindling of KFP -08d.

II. KFP1 Further Characterization

The above summarized asCP A148G mutant (designated KFP1) has remarkable properties, making it quite suitable for use as a photoactivated fluorescent label. Similar to wild-type asCP, KFP1 is a chromoprotein that is capable of kindling in intense green light and quenching in blue light. Initially nonfluorescent, kindled KFP1 becomes a brightly fluorescent protein with an excitation maximum at 580 nm and emission maximum at 600 nm.

KFP1 relaxes back to a nonfluorescent state with a half-life of about 50 seconds, which is approximately seven times longer than the wild-type protein. Furthermore, irradiation of KFP1 with green light of greater intensity and/or longer duration, i.e., an intensity of from about 1 W/cm2 to about 10⁶ W/cm² and a duration of from about 30 minutes to 0.001 second respectively, causes irreversible kindling. Irreversibly kindled KFP1 gives stable red fluorescence with an intensity at least 30 times brighter than the unkindled protein. The fluorescence of irreversibly kindled KFP1 does not fade for more than a year following the kindling event. In addition, irreversibly kindled KFP1 does not undergo significant quenching on irradiation with blue light.

Low intensity green light causes no kindling of KFP1 and can therefore be used as excitation light to visualize both reversibly and irreversibly kindled KFP1 without inducing kindling in the surroundings. This feature allows kindled objects to be tracked over long times without loss of image contrast. Moreover, the quenching property of KFP1 can be used to improve the contrast between the irreversibly kindled KFP1 and the surroundings.

III. Use of KFP for Cell Tracking—Embryonic Fate Mapping

The utility of KFP1 for cell tracking was demonstrated using embryonic fate mapping as an example. KFP1 cDNA was subcloned into the p35T vector. The resulting construct was digested with EcoRI Synthetic mRNA and was transcribed using a SP6 Message Machine kit (Ambion). The mRNA was purified with an RNeasy column (Qiagen) kit and microinjected into embryos (1 ng per blastomere). Embryos were staged according to the method of Nieuwkoop and Faber ((1967) Normal Table of Xenopus laevis (Daudin) (North-Holland, Amsterdam). KFP1 was kindled for 15 min using a TRITC filter set on a Polyvar microscope (Reihert-Jung) through a ×40 objective with a closed diaphragm. The embryo was photographed on a Leica MZIII fluorescent stereomicroscope with a TRITC filter set. A group of cells within the neural plate of the Xenopus laevis embryo was labeled. The trunk of the neural plate extends considerably in the anterior-posterior direction during neurulation due to the transversal intercalation of cells within this region. Keller (2000) Philos. Trans. R. Soc. 355, 897 (2000).

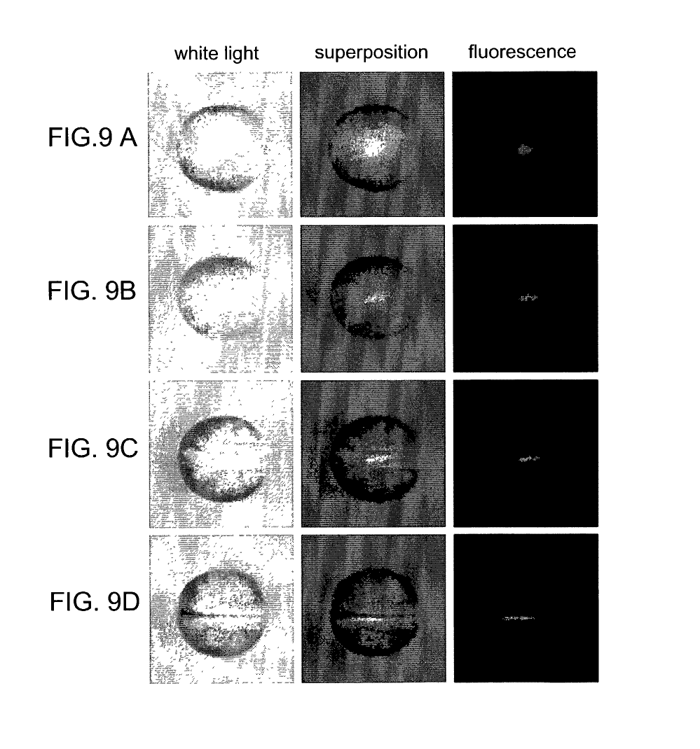

Animal poles of two-blastomere stage Xenopus embryos were microinjected with KFP1 mRNA. At the early neurula stage, a round-shaped group of cells within the neural plate of the embryo was irreversibly kindled by irradiation with intense green light (using a TRITC filter set on a Polyvar microscope (Reihert-Jung) through a ×40 objective, 100 watt lamp, 15 minutes) (FIG. 9A). Irradiated cells became brightly fluorescent and were tracked in the developing embryo. Longitudinal extension accompanied by transversal convergence of the labeled group of cells was visible after the first two hours of incubation (FIG. 9B). During further incubation, the anterior-posterior extension and transverse narrowing of the labeled spot became more pronounced (FIG. 9C). At the end of neurulation the labeled spot appeared as a thin stripe on the surface of the left neural fold (FIG. 9D).

This experiment demonstrates that KFPs provide precise fluorescent cell labeling and tracking of Xenopus embryo development. The approach employed here is therefore suitable for use in a wide range of systems, where fluorescent dyes are commonly used for tracking cell migration: e.g., for the study of morphogenesis, inflammation, and metastasis. The KFP-based technique reported here expands the possibilities for cell labeling by allowing precise laser beam fluorescent labeling in living tissues.

IV. Use of KFPs for Tracking Movement of Mitochondria

The utility of KFP1 for intracellular movement tracking was demonstrated by monitoring mitochondrial movement. The KFP1 sequence was subcloned (in place of ECFP) into pECFP-Mito vector (Clontech Laboratories Inc., Palo Alto, Calif.) containing a mitochondrial targeting sequence derived from the precursor of subunit VIII of human cytochrome C oxidase. The PC-12 cell line was transfected with the resulting construct using the Calcium Phosphate transfection system (Gibco). A Carl Zeiss Confocal Laser Scanning Microscope LSM 510 was used for KFP1 kindling, quenching and fluorescence monitoring. A HeNe laser line (543 nm, 1 mW) was employed as the light source for reversible kindling (5% power), irreversible kindling (30% power) and excitation (1% power). An Ar laser line (458 nm, 40 mW, 1% power) was used as the quenching light source.

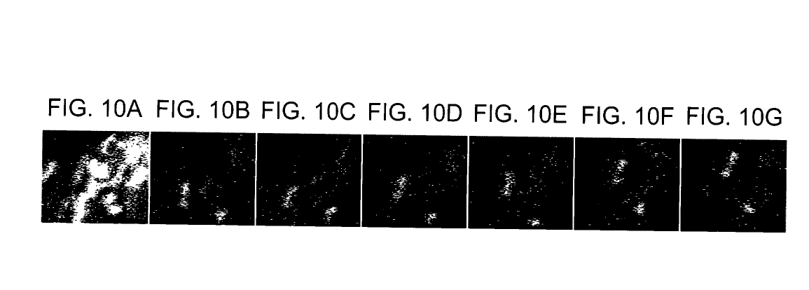

Mitochondria in the transfected cells remained nonfluorescent (no observed kindling) upon irradiation using a 1% power scanning green laser (once per 10 seconds). However, increasing the beam intensity to 5% power caused the mitochondria to brightly fluoresce for several minutes, indicating reversible kindling of KFP1 (FIG. 10A).

Brief irradiation (about 1 ms per pixel) with 30% power green laser light induced irreversible kindling of the KFP1 in all mitochondria within the irradiated field. These mitochondria remained brightly fluorescent indefinitely and could be observed by use of a 1% power green laser as the excitation light source (FIG. 10B-F).

Irradiation with weak (1% power) blue laser light caused instantaneous quenching of reversibly kindled mitochondria. In contrast, no significant quenching was observed for the irreversibly kindled mitochondria (compare FIGS. 10A and 10B).

Regardless of whether the kindling was reversible or irreversible, 1% power green laser light could be used to visualize the kindled mitochondria without inducing the kindling of other mitochondria within the visualized field. The technique described here demonstrates that kindling fluorescent proteins, such as KFP1, can be employed in directly tracking the migration of single mitochondria, and can therefore be employed in tracking the movement of other organelles and cell structures. Such investigations are of great interest in questions of the role of mitochondria in apoptosis, redistribution of subcellular structures during cell division, vesicular transport, axonal transport, etc.

It is evident from the above results and discussion that the subject invention provides an important new class of photoactivatable fluorescent proteins that can be employed as labels in a variety of different applications, including the cellular and subcellular component, e.g., protein and organelle, tracking applications. The use of kindling fluorescent proteins according to the subject invention in applications for which photobleaching is currently employed overcomes many of the disadvantages associated with photobleaching. The intensity of light needed to kindle a kindling fluorescent protein is substantially lower than the light intensities used in photobleaching. Use of a lower light intensity avoids damage to cells and cellular structures; therefore, the studies using kindling fluorescent proteins are less prone to artifact. Whereas photobleaching frequently disturbs the native conformation of a protein whose movement in a cell is being studied, a protein labeled with a kindling fluorescent protein is in its native conformation. Unlike with photobleaching techniques, the use of kindling fluorescent proteins allows direct monitoring of protein movement between and among cells, and in whole organisms. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A nucleic acid present in other than its natural environment, wherein said nucleic acid encodes a kindling fluorescent protein that goes from a first substantially non-fluorescent or non-fluorescent state to a second fluorescent state upon exposure to a kindling stimulus.

2. The nucleic acid according to claim 1, wherein said kindling stimulus is light of a kindling wavelength, intensity and duration effective to kindle said kindling fluorescent protein.

3. The nucleic acid according to claim 2, wherein said kindling wavelength of said kindling stimulus ranges from about 200 to 1500 nm.

4. The nucleic acid according to claim 3, wherein said kindling intensity of said kindling stimulus ranges from about 0.01 to about 10⁶W/cm².

5. The nucleic acid according to claim 4, wherein said kindling duration of said kindling stimulus ranges from about 1 millisecond to about 60 minutes.

6. The nucleic acid according to claim 1, wherein said kindling fluorescent protein does not have an amino acid sequence that is identical to SEQ ID NOs: 02 or 08.

7. The nucleic acid according to claim 1, wherein said kindling fluorescent protein is a mutant of a wild type kindling fluorescent protein.

8. The nucleic acid according to claim 1, wherein said kindling fluorescent protein is a wild type protein or mutant thereof from a non-bioluminescent Cnidarian species.

9. The nucleic acid according to claim 8, wherein said cnidarian species is an Anthozoan species.

10. The nucleic acid according to claim 1, wherein said second state is transient.

11. The kindling fluorescent protein of claim 1, wherein said second fluorescent state is permanent.

12. The nucleic acid according to claim 1, wherein said nucleic acid is isolated.

13. A fragment of a nucleic acid according to claim 1.

14. A construct comprising a vector and a nucleic acid according to claim 1.

15. An expression cassette comprising:
(a) a transcriptional initiation region functional in an expression host;
(b) a nucleic acid according to claim 1; and
(c) and a transcriptional termination region functional in said expression host.

16. A cell, or the progeny thereof, comprising an expression cassette according to claim 15 as part of an extrachromosomal element or integrated into the genome of a host cell.

17. A protein or fragment thereof encoded by a nucleic acid according to claim 1.

18. An antibody binding specifically to a protein according to claim 17.

19. A transgenic cell or the progeny thereof comprising a transgene that comprises a nucleic acid according to claim 1.

20. A transgenic organism comprising a transgene that comprises a nucleic acid according to claim 1.

21. A method of producing a kindling fluorescent protein, said method comprising:
growing a cell according to claim 16, whereby said kindling fluorescent protein is expressed.

22. The method according to claim 21, wherein said method further comprises isolating said kindling fluorescent protein substantially free of other proteins.

23. A method of producing a fluorescent protein, said method comprising:
subjecting a kindling fluorescent protein according to claim 17 to a kindling stimulus to produce a kindled kindling fluorescent protein which is fluorescent.

24. The method according to claim 23, wherein said kindling fluorescent protein is present inside of an organism.

25. The method according to claim 24, wherein said kindling fluorescent protein is present inside of a cell.

26. The method according to claim 23, wherein said kindling stimulus is light of a kindling wavelength, intensity and duration effective to kindle said kindling fluorescent protein.

27. The method according to claim 26, wherein said kindling wavelength of said kindling stimulus ranges from about 200 to about 1500 nm.

28. The method according to claim 26, wherein said kindling intensity of said kindling stimulus ranges from about 0.01 to about 10⁶W/cm².

29. The method according to claim 26, wherein said kindling duration of said kindling stimulus ranges from about 1 millisecond to about 60 minutes.

30. A method of detecting an entity in a composition, the method comprising:
(a) providing said entity as an entity labeled with a kindling fluorescent protein;
(b) kindling said kindling fluorescent protein label with a kindling stimulus to produce a kindled kindling fluorescent protein label; and
(c) exciting said kindled kindling fluorescent protein label with light and detecting any resultant fluorescence therefrom to detect said entity.

31. The method according to claim 30, wherein said kindling stimulus is light of a kindling wavelength, intensity and duration effective to kindle said kindling fluorescent protein.

32. The method according to claim 31, wherein said kindling wavelength of said kindling stimulus ranges from about 200 to about 1500 nm.

33. The method according to claim 31, wherein said kindling intensity of said kindling stimulus ranges from about 0.01 to about 10⁶W/cm².

34. The method according to claim 31, wherein said kindling duration of said kindling stimulus ranges from about 1 millisecond to about 60 minutes.

35. The method according to claim 30, wherein said entity is a protein.

36. The method according to claim 30, wherein said entity is an organelle.

37. The method according to claim 30, wherein said entity is a cell.

38. The method according to claim 30, wherein said composition is a cell.

39. The method according to claim 30, wherein said composition is a multicellular composition.

40. The method according to claim 39, wherein said multiceullular composition is a multicellular organism.

41. The method according to claim 30, wherein said method is a method of monitoring the movement of said entity.

42. A system for producing a kindled fluorescent protein from a kindling fluorescent protein, said system comprising:
(a) said kindling fluorescent protein or a nucleic acid encoding the same; and
(b) a source of a kindling stimulus.

43. A kit comprising a nucleic acid according to claim 1 and instructions for producing a fluorescent protein from said nucleic acid.
